(19) United States
(12) Patent Application Publication
(10) Pub. No.: US 2026/0275410 A1
(43) Pub. Date: Sep. 17, 2026

(54) METHODS FOR PREPARATION AND ANALYSIS OF PROXIMITY-LIGATED NUCLEIC ACIDS FROM SINGLE CELLS

(71) Applicants: Arima Genomics, Inc., Carlsbad, CA (US); New York University, New York, NY (US)

(72) Inventors: Anthony Schmitt, Holly Springs, NC (US); Soobeom Lee, New York, NY (US); Yohana Ghebrechristos, New York, NY (US); Iannis Aifantis, Brooklyn, NY (US)

(73) Assignees: Arima Genomics, Inc., Carlsbad, CA (US); New York University, New York, NY (US)

(21) Appl. No.: 18/878,838

(22) PCT Filed: Jun. 26, 2023
(86) PCT No.: PCT/US2023/069104

§ 371 Date: Dec. 24, 2024

Related U.S. Application Data
(60) Provisional application No. 63/355,774, filed on Jun. 27, 2022.

(51) Int. Cl. C12Q 1/6869; C12N 9/12; C12N 15/10; C12Q 1/6876; C40B 40/08; C40B 50/06; C40B 70/00

(52) U.S. Cl. CPC C12Q 1/6869; C12N 9/1276; C12N 15/1065; C12N 15/1082; C12Q 1/6876; C12Y 207/07049; C40B 40/08; C40B 50/06; C40B 70/00

(57) ABSTRACT

The technology relates in part to methods for preparation and analysis of proximity-ligated nucleic acids from single cells. This technology also relates in part to single-cell workflows for multiomic analyses of chromatin interactions, accessibility, gene expression, and protein expression.

### CROSS REFERENCE TO RELATED APPLICATIONS

This application claims the benefit under 35 U.S.C. § 119 (e) of U.S. provisional application No. 63/355,774 filed Jun. 27, 2022. The entire content of the referenced applications is incorporated by reference herein.

### FIELD

The technology relates in part to methods for preparation and analysis of proximity-ligated nucleic acids from single cells. This technology also relates in part to single-cell workflows for multiomic analyses of chromatin interactions, accessibility, gene expression, and protein expression.

### BACKGROUND

Normally, cells in a human carry the same DNA sequence, and yet, cell morphology and function in the context of cell-types, tissues & organs are vastly different. Cells achieve this diversity by expressing different sets of genes. Cells utilize regulatory elements encoded within the nucleoprotein structure of chromatin that instruct when and at what levels to express genes. Gene mis-regulation is a major cause of disease. Assays such as Transposase-Accessible Chromatin (ATAC) and Chromatin Immunoprecipitation (ChIP) followed by Nextgen Sequencing (NGS), commonly referred to as ATAC-Seq and ChIP-Seq respectively, are useful mapping tools to define regulatory elements by virtue of their capability to probe nucleosome positioning and chromatin accessibility (in the case of ATAC-Seq), and transcription factor occupancy (in the case of ChIP-Seq). The Cancer Genome Atlas (TCGA), and similar projects, have collectively generated>20,000 ATAC-Seq & ChIP-Seq datasets. However, obtaining a mechanistic view of gene regulation hinges on making two essential advancements: (1) evolving from the current one-dimensional (1D) to three-dimensional (3D) view of gene regulation; and (2) pushing such capability toward single-cell analysis. One deficit of the existing gene regulatory maps is a disregard for the 3D context of the genome. The biochemical steps used to map regulatory elements (via ATAC-Seq or ChIP-Seq) are typically performed on chromatin that is fragmented prior to preserving any 3D context. Every human cell contains 2 meters of DNA, that can be visualized as a bowl of spaghetti, is folded in 3D in a precise manner whose mis-folding can lead to mis-regulation & disease. Accordingly, an advancement from 1D gene regulation maps to a 3D view is useful for understanding cellular mechanisms that may be subject to therapeutic intervention. The next needed advancement is to push this capability of 3D gene regulation (3DGR) maps to single cells. Often, disease microenvironments manifest in heterogenous group of cells, each with its own regulatory programs, necessitating cell-by-cell profiling of 3DGR. Precision diagnosis and therapies, including gene and cell therapies, depend on mechanistic models of cellular function, differentiation, and diversity. Efforts toward building single cell 3DGR (herein described as either sc3DGR or Drop-C) maps can unravel these mechanisms.

### SUMMARY

Provided in certain aspects are methods for preparing nucleic acid from single cells and/or single cell nuclei, comprising a) contacting a population of cells and/or cell nuclei with a first crosslinking agent and a second crosslinking agent, thereby generating a population of double-crosslinked cells and/or double-crosslinked cell nuclei; b) contacting the population of cells and/or cell nuclei comprising double-crosslinked cells and/or double-crosslinked cell nuclei with one or more agents that preserve spatial-proximity relationships in the nucleic acid of the cells and/or cell nuclei; and c) partitioning the population of cells and/or cell nuclei into partitions comprising partitioned single cells and/or partitioned single cell nuclei.

Also provided in certain aspects are methods for preparing nucleic acid from single cells and/or single cell nuclei, comprising: a) contacting a population of cells and/or cell nuclei with one or more antibodies conjugated to an oligonucleotide; b) contacting the population of cells and/or cell nuclei with a first crosslinking agent and a second crosslinking agent, thereby generating a population of double-crosslinked cells and/or double-crosslinked cell nuclei, wherein the first crosslinking agent comprises formaldehyde and the second crosslinking agent comprises disuccinimidyl glutarate (DSG); c) contacting the population of cells and/or cell nuclei comprising double-crosslinked cells and/or double-crosslinked cell nuclei with one or more agents that generate proximity ligated nucleic acid molecules in the nucleic acid of the cells and/or cell nuclei, wherein the one or more reagents comprise two or more restriction endonucleases and a ligase; d) contacting the population of cells and/or cell nuclei with one or more agents that enrich for accessible chromatin, wherein the one or more agents comprise a Tn5 transposase and one or more oligonucleotides comprising sequencing primer sequences; e) partitioning the population of cells and/or cell nuclei into droplet partitions comprising partitioned single cells and/or partitioned single cell nuclei, wherein the partitioning comprises contacting the population of cells and/or cell nuclei with gelbead-in-emulsion (GEM) and one or more cell-specific and/or nucleus-specific barcode oligonucleotide species; f) performing a heated crosslink reversal process and contacting cells and/or cell nuclei in the one or more partitions with an agent comprising a reverse transcriptase activity, thereby generating cDNA from mRNA present in the cells and/or cell nuclei in the one or more partitions; and g) generating one or more nucleic acid libraries from one or more of the cells and/or cell nuclei in the partitions, wherein the one or more nucleic acid libraries comprise: i) library molecules comprising accessible chromatin fragments; ii) library molecules comprising accessible chromatin fragments and proximity ligated chromatin fragments; iii) library molecules comprising oligonucleotides representing protein expression; and iv) library molecules comprising cDNA fragments representing gene expression. Also provided in certain aspects are kits comprising a) a first crosslinking agent; b) a second crosslinking agent; and c) one or more agents that preserve spatial-proximity relationships in nucleic acid of cells and/or cell nuclei.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows an embodiment of a single-cell 3D gene regulation (sc3DGR)/Drop-C workflow. In this example, a sc3DGR workflow is carried out in bulk phase (top panel) followed by single-cell phase (bottom panel). Top panel: Step 1a includes an optional incorporation of a single-cell protein (scProtein) modality; cells are stained using oligo-conjugated antibodies (Abs). Cells are then universally crosslinked. Steps 1b-1d include chromatin conditioning, digestion, and proximity ligation. To incorporate a single-cell Transposase-Accessible Chromatin (scATAC) modality and to enable a single-cell High-throughput chromatin capture (scHiC) modality, chromatin is tagmented within cells using transposase (10X Genomics (10XG) kit). Polyadenylated mRNA is retained within the cells through Step1. Bottom panel: Step 2-cells are encapsulated and barcoded in gel bead-in-emulsions (GEMs) via 10XG Chromium instrument. The scHIC, scATAC, and scProtein modalities are barcoded using 10XG scATAC kit reagents, and the scRNA modality is barcoded using the 10XG Multiome kit reagents. Step 3-next generation sequencing (NGS) libraries are constructed. Depicted are NGS library molecules for each modality. For each library molecule type, the outer bars are sample-index containing NGS adapters, and the second bar from the left bar is a 10X barcode. For the accessible chromatin library molecule, the insert is an accessible chromatin fragment for scATAC. For the chromatin interaction library molecule, the chimeric insert is a chromatin interaction from an accessible region for scHiC. For the protein expression library molecule, the second bar from the right is an antibody derived tag (ADT) for scProtein. For the gene expression library molecule, the second bar from the right is cDNA, the third bar from the right is poly (dT), and the fourth bar from the right is a 10X unique molecular identifier (UMI) for scRNA.

### Step 4—Paired-End NGS.

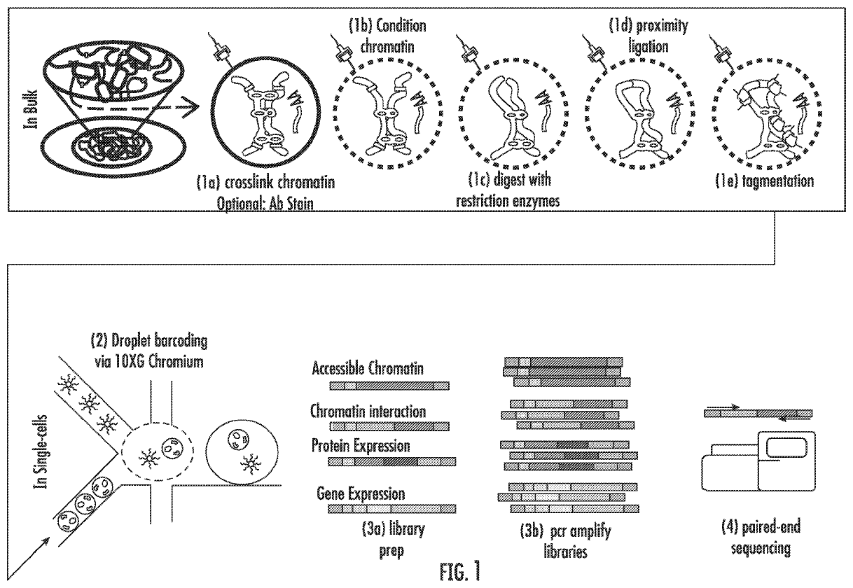

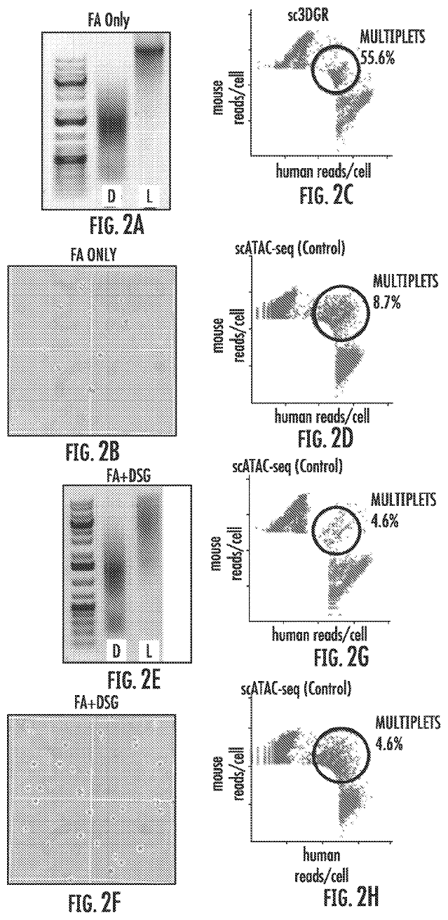

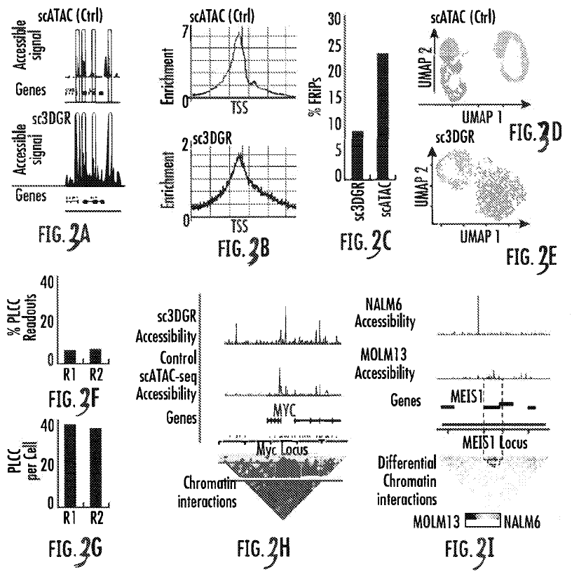

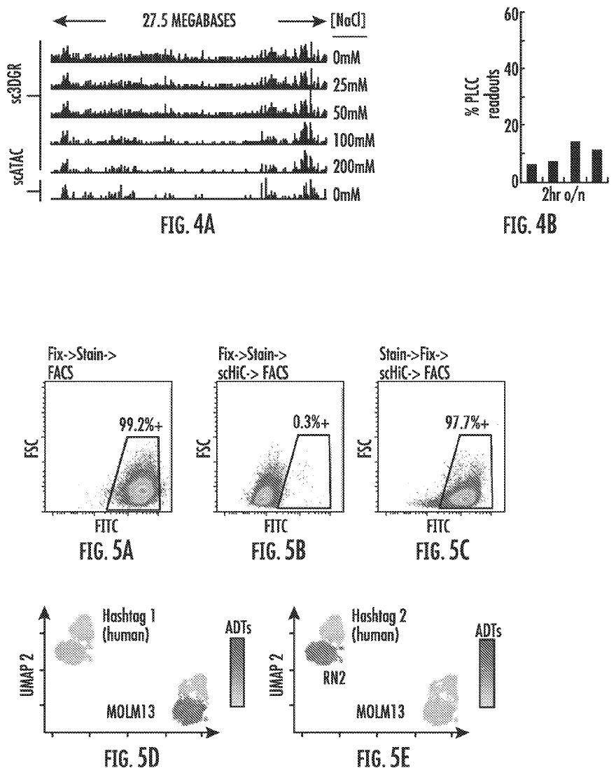

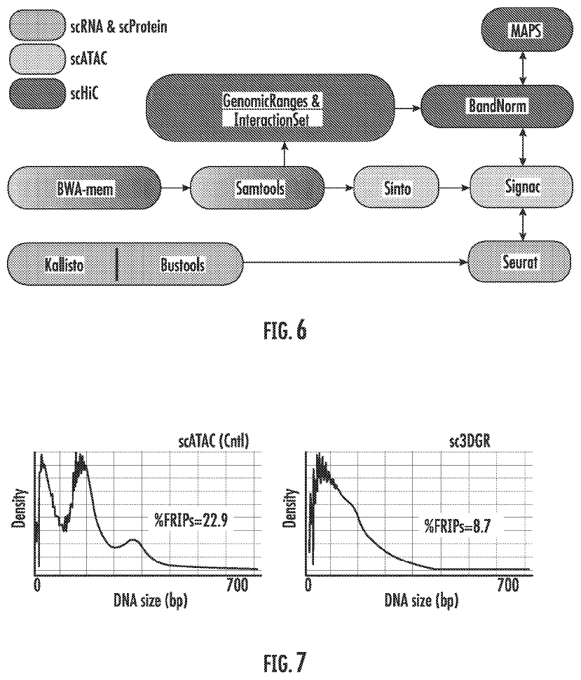

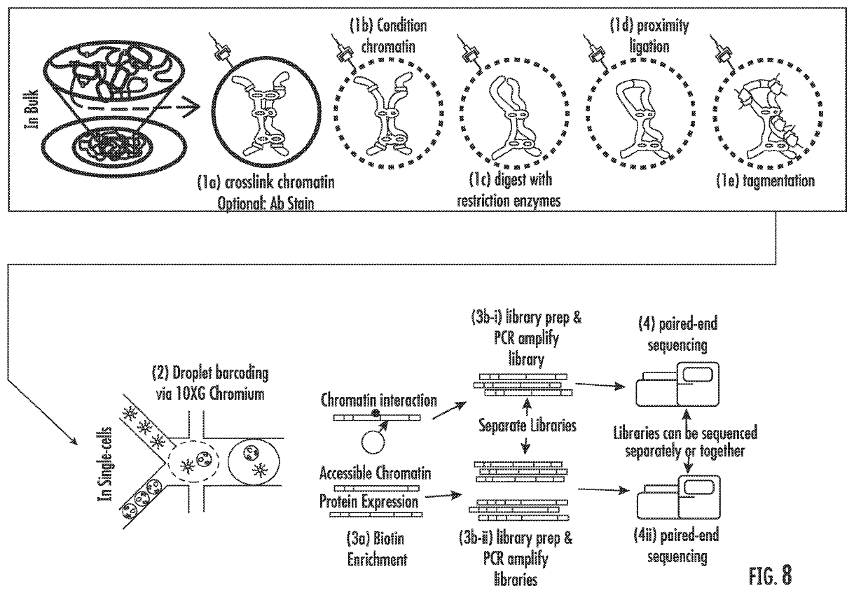

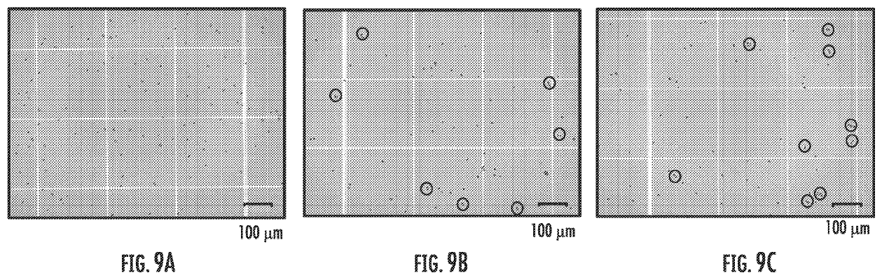

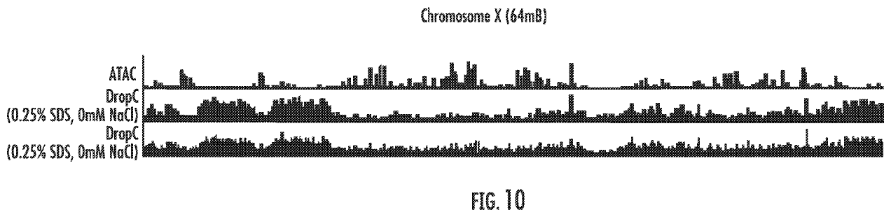

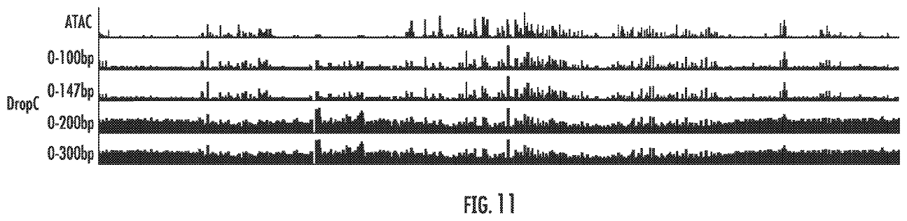

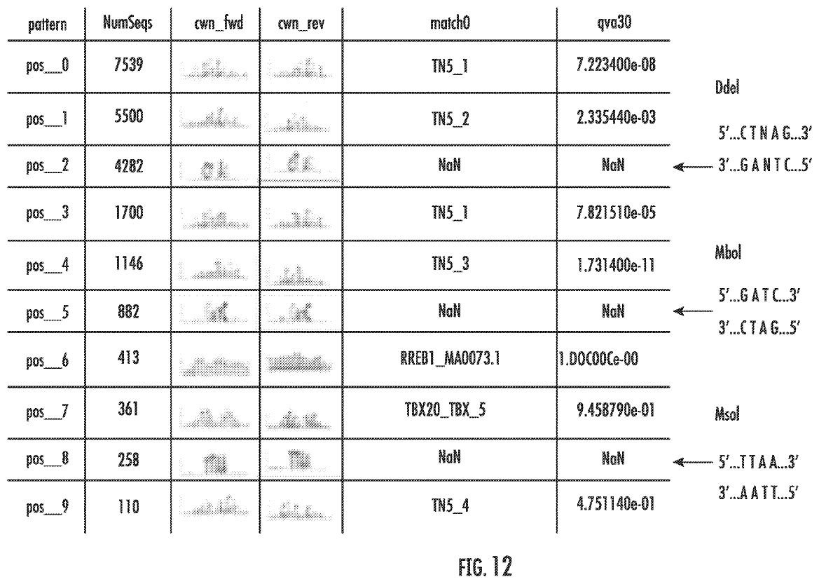

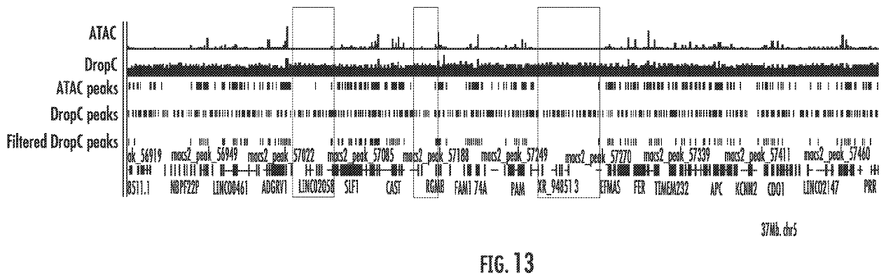

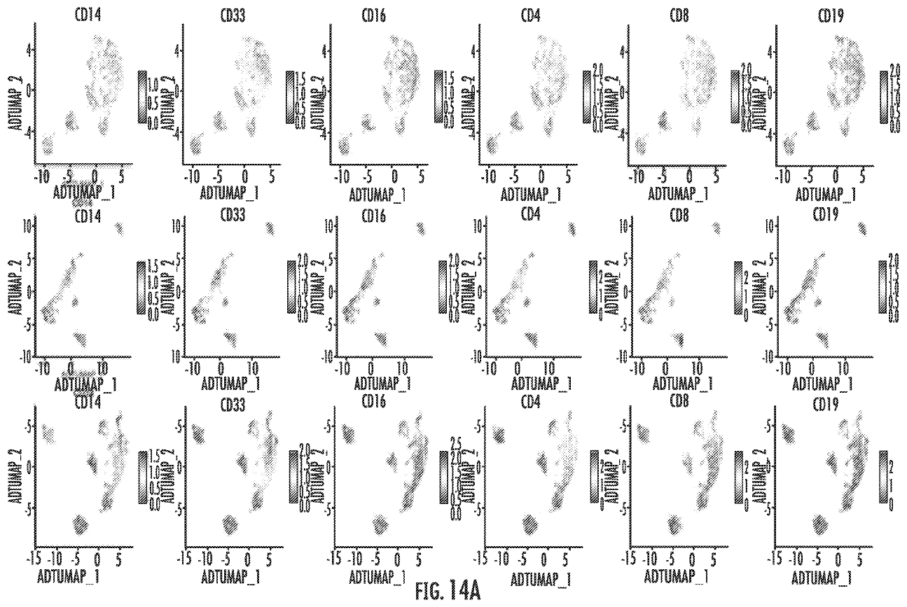

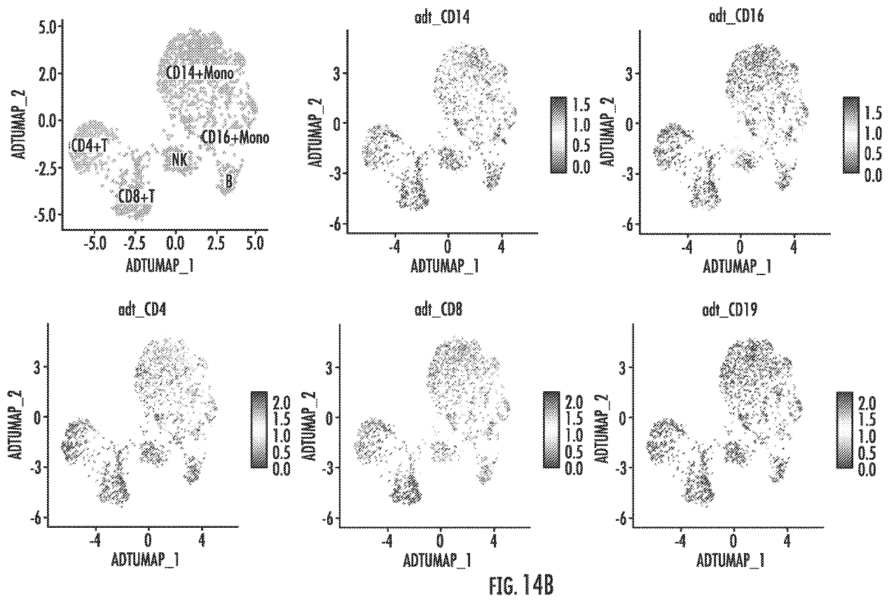

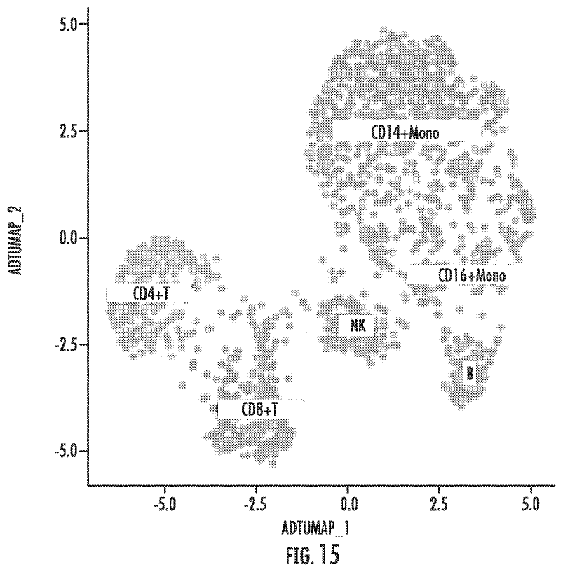

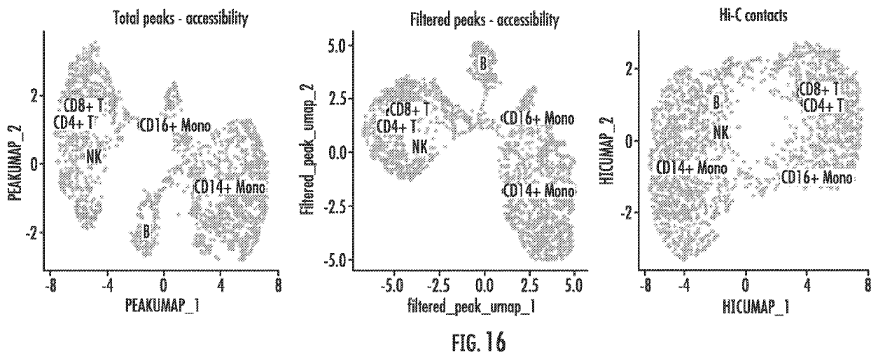

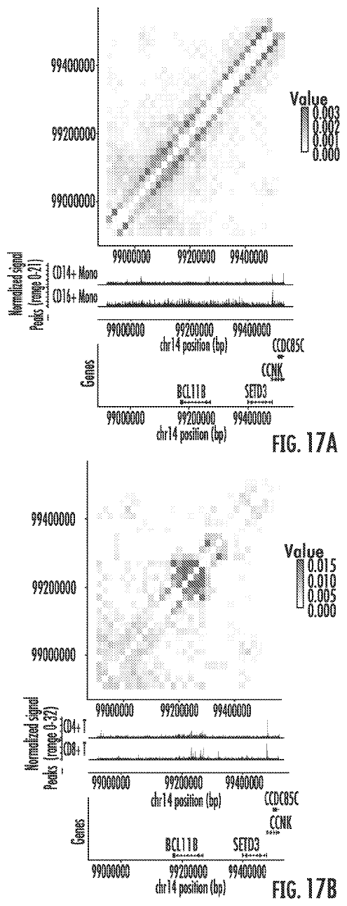

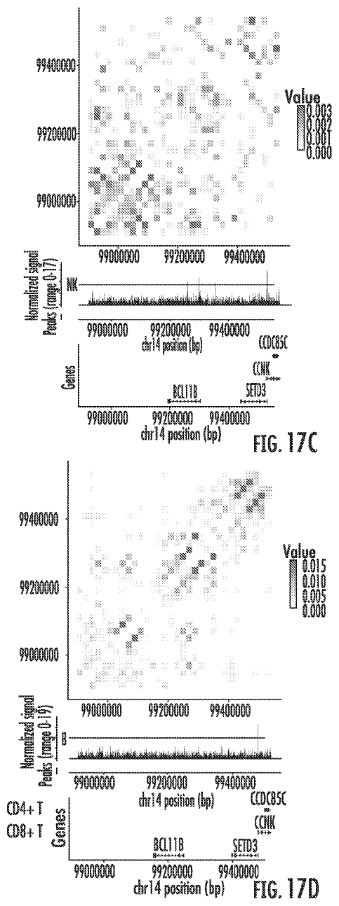

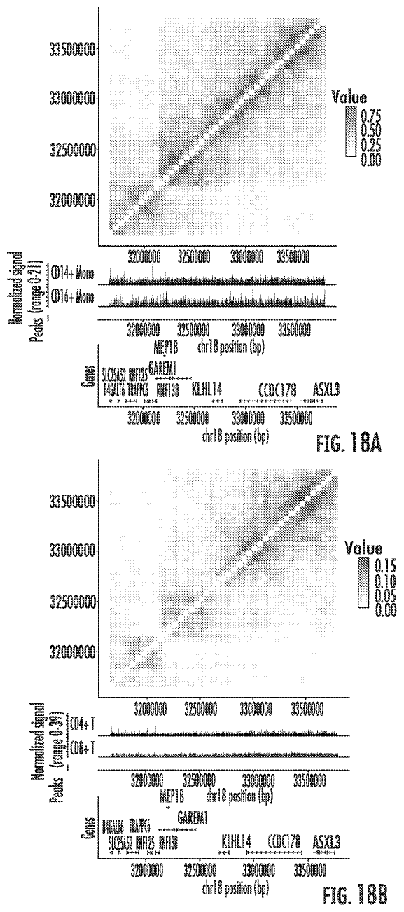

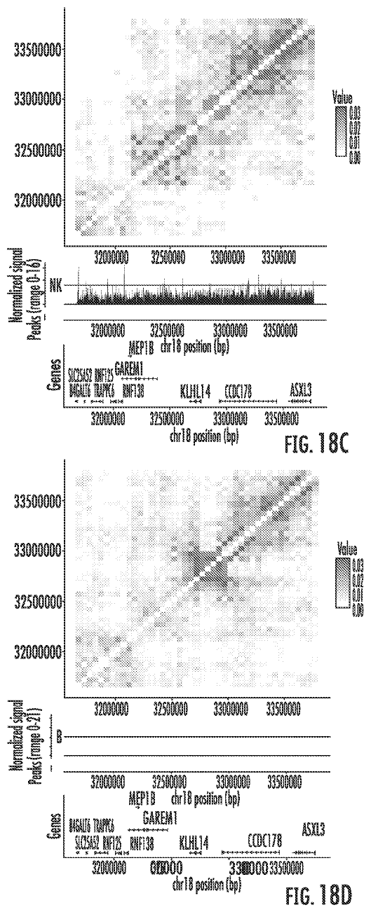

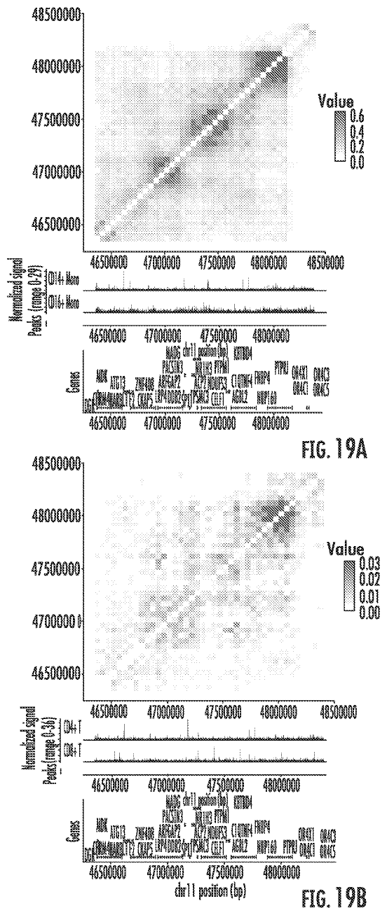

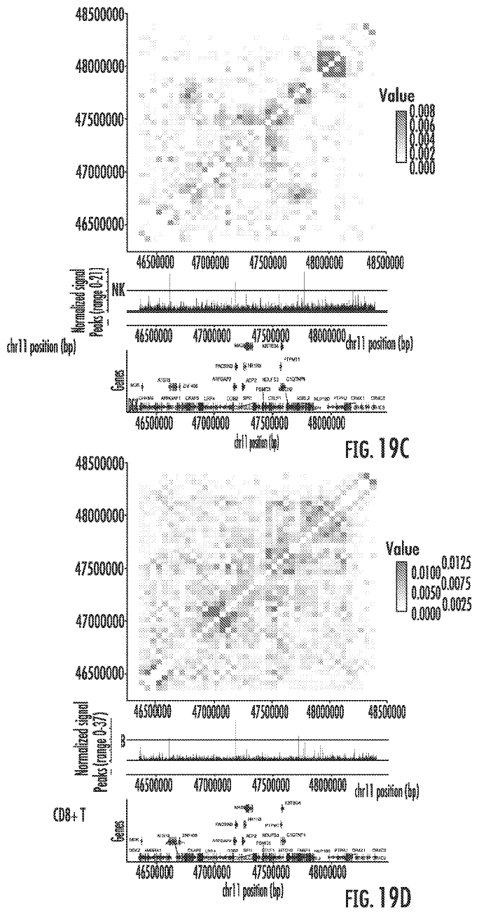

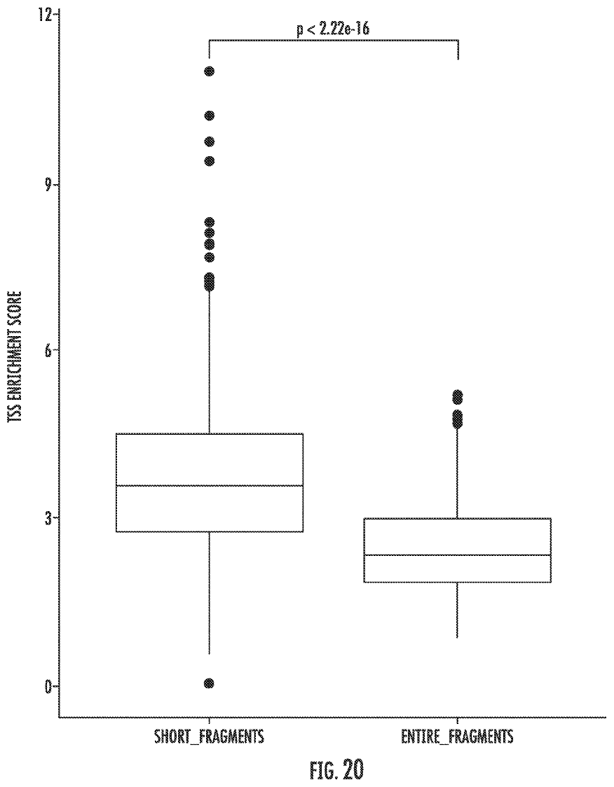

FIG. 2 shows a multiplet rate problem in sc3DGR and a dual crosslinking solution. Panel A: Gel electrophoresis of digested and ligated chromatin from a sc3DGR workflow on formaldehyde-fixed (“FA Only”) cells. Panel B: Representative light microscopy of proximity-ligated cells (FIG. 1, Step 1d). Crumpled/clumped cells were observed. Panel C: sc3DGR NGS analysis from formaldehyde fixed cell populations comprising a 50/50% mixture of human/mouse leukemia cells, showing the number of reads aligning to human (x-axis) and mouse (y-axis) for each cell barcode. The % of cell barcodes with reads mapping to mouse and human is shown (circled). Panel D: Same as panel C, except analysis of control scATAC-seq data. Panels E, F, G, and H mirror panels A, B, C, and D respectively, except using cells crosslinked sequentially with formaldehyde and DSG (“FA+DSG”).

FIG. 3 shows sc3DGR analyses of chromatin accessibility and chromatin interactions. Panel A: Chromatin accessibility signal in MOLM13 human leukemia cell line from scATAC-seq (control, top) and sc3DGR (bottom) around the MYC locus. Highlighted in vertical bars are the 5 highest accessibility “peaks” in the control scATAC-seq data, which correspond to the 5 highest “peaks” in the sc3DGR data. Panel B: Metaplot enrichment analysis of control scATAC-seq reads (top) and sc3DGR reads (bottom) piled up at transcription start sites (TSS). Panel C: Bar plot showing the % of readouts overlapping known ATAC peaks from the sc3DGR and control scATAC-seq data. Panels D and E: UMAP analysis using Signac applied to chromatin accessibility signal for each cell from the control scATAC-seq data (Panel D) and the sc3DGR data (Panel E). Panel F: Bar plot showing the % PLCC readouts from sc3DGR data in 2 human leukemia cell lines. Panel G: Bar plot showing the PLCC per cell from sc3DGR data in 2 human leukemia cell lines. Panel H: Snapshot of chromatin accessibility and chromatin interaction maps from single-cell resolved 3DGR data in MOLM13 cells. Top is chromatin accessibility signal from the sc3DGR data, middle is chromatin accessibility from control scATAC-seq data and bottom is chromatin interaction map derived from the scHiC modality of the sc3DGR data, showing presence of 2 adjacent 3D neighborhoods at MYC. Panel I: Snapshot at MEIS1 showing MOLM13-specific chromatin accessibility signal from single-cell resolved 3DGR data (top 2 tracks). Below is differential chromatin interaction map made by subtracting MOLM13 chromatin interactions from NALM6, and shows MOLM13-specific 3D chromatin neighborhood at MEIS1.

FIG. 4 shows improving scATAC and scHiC modalities in sc3DGR. Panel A: sc3DGR was performed on 5 replicates of MOLM13 cells. Cells were tagmented with Tn5 (FIG. 1, Step 1e) using increasing concentrations of NaCl from 0 mM (standard) to 200 mM. After tagmentation, libraries were droplet-barcoded and sequenced via deep NGS. As a control, a scATAC-seq library was prepared with standard conditions (0 mM NaCl) and sequenced. Reads were mapped and the NGS coverage was plotted along ~27.5 Mb region of chrX. Panel B: sc3DGR was performed in replicate using 2 hr or overnight dual restriction enzyme digestion (FIG. 1, Step 1c). After tagmentation, libraries were barcoded and sequenced via shallow NGS. Bar plot shows % PLCC readouts.

FIG. 5 shows development of scProtein modality in sc3DGR. Panel A: Cells were crosslinked, stained using anti-human hashtag 1 antibody conjugated to FITC (Biolegend), and then subject to FACS analysis gating on forward scatter (FSC, y-axis) and FITC signal (x-axis). Panel B: Cells were crosslinked, stained and then subject to HiC prior to FACS analysis. Panel C: Cells were stained, crosslinked, and then subject to HiC prior to FACS analysis. Panel D: Clustering analysis based on the scATAC modality from sc3DGR experiment on human/mouse mixture deep NGS. Overlaid are the antibody-derived tags (ADTs) from the human hashtag 1, of which>95% are derived from the correct sample (MOLM13). Panel E: Same as panel D, except mouse hashtag-2 ADTs assigned to RN2 cells.

FIG. 6 shows an example sc3DGR bioinformatics workflow. A sc3DGR bioinformatics workflow is shown for integrative processing of data types from each modality. Tools that are modality-specific are shown in solid-shading, and tools used for multiple modalities are shown in gradient-shading. The entire workflow is containerized via Docker and Singularity.

FIG. 7 shows quality control (QC) data for an scATAC modality in sc3DGR. NGS library size analysis for successful (22.9% FRIPs) scATAC-seq control library with nucleosome banding pattern (left), and moderately successful (8.7% FRIPs) sc3DGR library lacking the nucleosome banding pattern.

FIG. 8 shows a schematic of one embodiment of a single-cell 3D gene regulation (sc3DGR) workflow.

FIG. 9 shows images of multiplets (cell clumping) formed during one embodiment of a single-cell 3D gene regulation (sc3DGR) workflow.at varying concentrations of SDS.

FIG. 10 shows Manhattan plots comparing various salt concentrations used in scATAC and scHiC modalities in one embodiment of the sc3DGR workflow using peripheral blood mononuclear cells (PBMCs).

FIG. 11 shows Manhattan plots comparing various length of library molecules used in one embodiment of the sc3DGR workflow using peripheral blood mononuclear cells (PBMCs).

FIG. 12 shows an analysis of sequence motifs enriched at the transposase insertion sites that are outside of the true positive accessible regions.

FIG. 13 shows Manhattan plots of scATAC and scHiC modalities in one embodiment of the sc3DGR workflow where the scHiC is also subjected to filtering.

FIG. 14 shows clustering of PBMCs using cell-surface protein expression profiles in one embodiment of the sc3DGR workflow.

FIG. 15 shows clustering of PBMCs using cell-surface protein expression profiles in one embodiment of the sc3DGR workflow.

FIG. 16 shows clustering of PBMCs using cell-surface protein expression profiles in one embodiment of the sc3DGR workflow.

FIG. 17 shows HiC heat maps of chromatin interactions of the BCL11B encoding region in monocytes, T-cells, NK, and B-cells in accordance with one embodiment of the sc3DGR workflow.

FIG. 18 shows HiC heat maps of chromatin interactions of the KLHL14 encoding region in monocytes, T-cells, NK, and B-cells in accordance with one embodiment of the sc3DGR workflow.

FIG. 19 shows HiC heat maps of chromatin interactions of the SPI1 encoding region in monocytes, T-cells, NK, and B-cells in accordance with one embodiment of the sc3DGR workflow.

FIG. 20 shows a TSS metaplot of the data shown in FIG. 11.

### DETAILED DESCRIPTION

Provided herein are methods for preparing nucleic acid from single cells and/or single cell nuclei. Methods may include one or more modalities chosen from preserving nucleic acid spatial-proximity relationships in a population of cells and/or cell nuclei, enriching for accessible chromatin, assessing protein expression, assessing gene expression, and partitioning the population of cells and/or cell nuclei into single cells and/or single cell nuclei. In certain embodiments, methods may include preserving nucleic acid spatial-proximity relationships in a population of cells and/or cell nuclei and partitioning the population of cells and/or cell nuclei into single cells and/or single cell nuclei. In certain embodiments, methods may include preserving nucleic acid spatial-proximity relationships in a population of cells and/or cell nuclei, enriching for accessible chromatin, and partitioning the population of cells and/or cell nuclei into single cells and/or single cell nuclei. In certain embodiments, methods may include preserving nucleic acid spatial-proximity relationships in a population of cells and/or cell nuclei, enriching for accessible chromatin, assessing protein expression, and partitioning the population of cells and/or cell nuclei into single cells and/or single cell nuclei. In certain embodiments, methods may include preserving nucleic acid spatial-proximity relationships in a population of cells and/or cell nuclei, enriching for accessible chromatin, assessing gene expression, and partitioning the population of cells and/or cell nuclei into single cells and/or single cell nuclei. In certain embodiments, methods may include preserving nucleic acid spatial-proximity relationships in a population of cells and/or cell nuclei, enriching for accessible chromatin, assessing protein expression, assessing gene expression, and partitioning the population of cells and/or cell nuclei into single cells and/or single cell nuclei. Methods herein may further include generating double-crosslinked cells, double-crosslinked nuclei, and/or double-crosslinked chromatin in a population of cells and/or cell nuclei. Methods herein may further include generating one or more nucleic acid libraries (e.g., one or more sequencing libraries).

### Spatial-Proximity Relationships

In some embodiments, a method herein comprises a process that preserves spatial-proximity relationships (e.g., spatial-proximal contiguity; spatial-proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi: 10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi: 10.1038/nrm.2016.104; each of which is incorporated by reference in its entirety, to the extent permitted by law)).

Methods herein may include contacting a population of cells and/or cell nuclei with one or more agents that preserve spatial-proximity relationships in the nucleic acid of the cells and/or cell nuclei. Agents that preserve spatial-proximity relationships generally refer to agents used in methods that capture and preserve the native spatial conformation exhibited by nucleic acids when associated with proteins as in chromatin and/or as part of a nuclear matrix. Spatial-proximity relationships may be preserved by any suitable method including, but not limited to, proximity ligation, solid substrate-mediated proximity capture (SSPC), compartmentalization with or without a solid substrate, and/or use of a Tn5 tetramer. Methods that preserve spatial-proximity relationships may be based on proximity ligation or may be based on a different principle where spatial proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where special proximity is inferred based on a principle other than proximity ligation may include, for example, SPRITE, scSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a method herein comprises generating proximity ligated nucleic acid molecules (e.g., using a method described herein). In some embodiments, a method herein comprises sequencing the proximity ligated nucleic acid molecules, e.g., by a suitable sequencing process known in the art or described herein. In some embodiments, nucleic acid molecules may be fragmented and sequenced using short-read sequencing methods (e.g., Illumina, nucleic acid fragments of lengths approximately 500 base pairs). In some embodiments, intact nucleic acid molecules can be sequenced using long-read sequencing (e.g., Illumina, Oxford Nanopore, or others, nucleic acid fragments of lengths approximately 30 kilobases or greater).

In some embodiments, methods that preserve spatial-proximity relationships comprise methods that generate proximity ligated nucleic acid molecules (e.g., using proximity ligation). In some embodiments, a method herein comprises contacting the population of cells and/or cell nuclei with one or more reagents that generate proximity ligated nucleic acid molecules. A proximity ligation method is one in which natively occurring spatially proximal nucleic acid molecules are captured by ligation to generate ligated products. Proximity ligation methods generally capture spatial-proximity relationships in the form of ligation products, whereby a ligation junction is formed between two natively spatially proximal nucleic acids. Once the ligation products are formed, the spatial-proximity relationship may be detected using a suitable sequencing method (e.g., next generation sequencing), whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules may include one or more reagents chosen from a restriction endonuclease (i.e., restriction enzyme), a DNA polymerase, a plurality of nucleotides comprising at least one labeled nucleotide (e.g., biotinylated nucleotide), and a ligase. In certain embodiments, two or more restriction endonucleases are used.

Cells and/or cell nuclei subjected to a proximity ligation process may form proximally ligated chromatin contacts (PLCC). The term proximally ligated chromatin contacts (PLCC) generally refers to proximity ligation events between two or more natively occurring spatially proximal nucleic acid molecules. Proximally ligated chromatin contacts (PLCC) may be quantified (e.g., via next generation sequencing and analysis of libraries prepared from DNA from cell(s) that have undergone a proximity ligation process), and such quantification may be used as a measure of performance of a proximity ligation assay. For example, a PLCC quantification may include a measure of PLCC per cell and/or cell nucleus. In some embodiments, a method herein comprises determining an amount of proximity ligated chromatin contacts (PLCC) per cell and/or cell nucleus. For example, in FIG. 1, the DNA sequencing libraries prepared and sequenced are barcoded in such a way that every library molecule, which includes molecules representing PLCCs (labeled in the figure as ‘chromatin interaction’ in step 3a), can be assigned to its cell of origin. After sequencing and analyzing a sc3DGR library, PLCCs are obtained with cellular barcodes, and the detected number of PLCCs derived from each individual cell are quantified, providing an average measure of PLCCs per cell. In some embodiments, a proximity ligation method herein generates between about 1,000 or more to about 10,000 or more PLCC per cell and/or cell nucleus. For example, a proximity ligation method herein may generate greater than 1,000, 2,000, 3,000, 4,000, 5,000, 6,000, 7,000, 8,000, 9,000, or 10,000 PLCC per cell and/or cell nucleus. In some embodiments, a proximity ligation method herein generates greater than 5,000 PLCC per cell and/or cell nucleus. In certain instances, a PLCC quantification may include a percentage or ratio (e.g., percentage of PLCC readouts; ratio of PLCC to non-PLCC) of PLCC, often assessed at the sequence read level. In some embodiments, a method herein comprises determining a percentage of proximity ligated chromatin contacts (PLCC). For example, when the modalities analyzed are scATAC and scHiC, a portion of the DNA library molecules and sequencing readouts contain a PLCC (labeled as ‘chromatin interaction’ in FIG. 1, step 3a), and a portion will not (labeled as ‘accessible chromatin’ in FIG. 1 step 3a). Accordingly, percentage of PLCC readouts can be defined as the number of sequenced molecules that comprise a PLCC divided by the total number of sequencing readouts. In some embodiments, a proximity ligation method herein generates a percentage of PLCC between about 5% or more to about 50% or more. For example, a proximity ligation method herein may generate a percentage of PLCC greater than about 5%, 10%, 15%, 20%, 25%, 30%, 35%, 40%, 45%, or 50%. In some embodiments, a proximity ligation method herein generates a percentage of PLCC greater than about 30%. In some embodiments, a proximity ligation method herein generates a percentage of PLCC greater than about 40%. In some embodiments, a proximity ligation method herein comprises contacting a population of cells and/or cell nuclei with one or more restriction endonucleases. In some embodiments, a proximity ligation method herein comprises contacting a population of cells and/or cell nuclei with two or more restriction endonucleases. Restriction endonucleases may be chosen from type I, Il or Ill restriction endonucleases such as Accl, Acil, Afllll, Alul, Alw441, Apal, Asnl, Aval, Avall, BamHI, Banll, Bcll, Bgll, Bglll, Binl, Bsml, BssHII, BstEll, BstUI, Cfol, Clal, Ddel, Dpnl, Dpnll, Dral, EcIXI, EcoRI, EcoRI, EcoRII, EcoRV, Haell, Haell, Hhal, Hindll, Hindlll, Hpal, Hpall, Kpnl, Kspl, Maell, McrBC, Mlul, MluNI, Mspl, Ncil, Ncol, Ndel, Ndell, Nhel, Notl, Nrul, Nsil, Pstl, Pvul, Pvull, Rsal, Sacl, Sall, Sau3Al, Scal, ScrFI, Sfil, Smal, Spel, Sphl, Sspl, Stul, Styl, Swal, Taql, Xbal, and Xhol. In some embodiments, a restriction endonuclease is chosen from one or more of Mbol, Hinfl, Msel and Ddel. In some embodiments, a restriction endonuclease is chosen from one or more of HpyCH4IV, Hinfl, HinP11 and Msel. In some embodiments, a restriction endonuclease is Nlalll. In some embodiments, a restriction endonuclease is chosen from one or more of Acil, HinP11, Hpall, HpvCH4IV. Msol, and Taal. In some embodiments, a restriction endonuclease is chosen from one or more of Bfal, Msel, and CviQI. In some embodiments, a restriction endonuclease is chosen from one or more of LlaAl, Mbol, Mgol, MkrAl, Ndell, Nlall, NmeCI, Nphl, Sau3AI, Kzo91, Dpnll, BstMBI, BssMI, and Bsp1431. In some embodiments, a restriction endonuclease is Dpnll. In some embodiments, a restriction endonuclease is Hinfl.

Contacting a population of cells and/or cell nuclei with one or more restriction endonucleases typically generates nucleic acid fragments of varying size (i.e., length). In some embodiments, contacting a population of cells and/or cell nuclei with one or more restriction endonucleases generates nucleic acid fragments with an average, mean, or median size of about 200 base pairs to about 1000 base pairs. For example, contacting a population of cells and/or cell nuclei with one or more restriction endonucleases may generate nucleic acid fragments with an average, mean, or median size of about 200 base pairs, 300 base pairs, 400 base pairs, 500 base pairs, 600 base pairs, 700 base pairs, 800 base pairs, 900 base pairs, or 1,000 base pairs. In some embodiments, contacting a population of cells and/or cell nuclei with one or more restriction endonucleases generates nucleic acid fragments with an average, mean, or median size of about 800 base pairs.

Cells and/or cell nuclei may be contacted with one or more restriction endonucleases for a suitable duration of time. For example, cells and/or cell nuclei may be contacted with one or more restriction endonucleases for a duration of time suitable to generate a desired proximity ligation product (e.g., having a certain PLCC per cell and/or nucleus or PLCC readout percentage). In some embodiments, a method herein comprises contacting a population of cells and/or cell nuclei with one or more restriction endonucleases for about 2 hours or more. In some embodiments, a method herein comprises contacting a population of cells and/or cell nuclei with one or more restriction endonucleases for more than 2 hours. For example, a method herein may comprise contacting a population of cells and/or cell nuclei with one or more restriction endonucleases for about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, a method herein comprises contacting a population of cells and/or cell nuclei with one or more restriction endonucleases for more than 8 hours. In some embodiments, a method herein comprises contacting a population of cells and/or cell nuclei with one or more restriction endonucleases overnight (e.g., about 8-12 hours).

In some embodiments, a population of cells and/or cell nuclei is contacted with one or more restriction endonucleases for a duration of time whereby the PLCC per cell and/or nucleus or PLCC readout percentage is increased compared to a population of cells and/or cell nuclei contacted with one or more restriction endonucleases for a shorter duration of time. In some embodiments, a population of cells and/or cell nuclei is contacted with one or more restriction endonucleases for a duration of time whereby the PLCC readout percentage is doubled compared to a population of cells and/or cell nuclei contacted with one or more restriction endonucleases for a shorter duration of time. In some embodiments, a population of cells and/or cell nuclei is contacted with two or more restriction endonucleases for a duration of time whereby the PLCC per cell and/or nucleus or PLCC readout percentage is increased compared to a population of cells and/or cell nuclei contacted with two or more restriction endonucleases for a shorter duration of time. In some embodiments, a population of cells and/or cell nuclei is contacted with two or more restriction endonucleases for a duration of time whereby the PLCC readout percentage is doubled compared to a population of cells and/or cell nuclei contacted with two or more restriction endonucleases for a shorter duration of time.

In some embodiments, a proximity ligation method herein comprises contacting a population of cells and/or cell nuclei with an agent comprising a ligase activity. Ligase activity may include, for example, blunt-end ligase activity, nick-sealing ligase activity, sticky end ligase activity, circularization ligase activity, cohesive end ligase activity, DNA ligase activity, RNA ligase activity, single-stranded ligase activity, and double-stranded ligase activity. Ligase activity may include ligating a 5′ phosphorylated end of one polynucleotide to a 3′ OH end of another polynucleotide (5′P to 3′OH). Ligase activity may include ligating a 3′ phosphorylated end of one polynucleotide to a 5′ OH end of another polynucleotide (3′P to 5′OH). In some embodiments, a method herein comprises contacting a population of cells and/or cell nuclei with a ligase. Suitable reagents (e.g., ligases) and kits for performing ligation reactions are known and available. Ligases that may be used include but are not limited to, T3 ligase, T4 DNA ligase, T7 DNA Ligase, E. coli DNA Ligase, Electro Ligase®, RNA ligases, T4 RNA ligase 1, T4 RNA ligase 2, SplintR® Ligase, RtcB ligase, Taq ligase, and the like and combinations thereof.

In some embodiments, reagents that generate proximity ligated nucleic acid molecules may include one or more polymerases (e.g., DNA polymerases). Any suitable polymerase may be used including, e.g., DNA polymerase I, TAQ DNA polymerase; E. coli DNA polymerase I, large (Klenow) fragment of DNA polymerase I, T4 DNA polymerase, Bacillus stearothermophilus (Bst) DNA polymerase, thermostable DNA polymerases (e.g., from hyperthermophilic marine Archaea), 9°N™ DNA Polymerase (GENBANK accession no. AAA88769.1), THERMINATOR polymerase (9°N™ DNA Polymerase with mutations: D141A, E143A, A485L), and the like. In some embodiments, a strand displacing polymerase is used (e.g., Bst DNA polymerase).

In some embodiments, reagents that generate proximity ligated nucleic acid molecules may include one or more labeled nucleotides. A labeled nucleotide may comprise a member of a binding pair. Binding pairs may include, for example, biotin/avidin, biotin/streptavidin, antibody/antigen, antibody/antibody, antibody/antibody fragment, antibody/antibody receptor, antibody/protein A or protein G, hapten/anti-hapten, folic acid/folate binding protein, vitamin B12/intrinsic factor, chemical reactive group/complementary chemical reactive group, digoxigenin moiety/anti-digoxigenin antibody, fluorescein moiety/anti-fluorescein antibody, steroid/steroid-binding protein, operator/repressor, nuclease/nucleotide, lectin/polysaccharide, active compound/active compound receptor, hormone/hormone receptor, enzyme/substrate, oligonucleotide or polynucleotide/its corresponding complement, the like or combinations thereof. In some embodiments, a labeled nucleotide comprises biotin.

In some embodiments, a labeled nucleotide comprises a first member of a binding pair (e.g., biotin); and a second member of a binding pair (e.g., streptavidin) is conjugated to a solid support or substrate. A solid support or substrate can be any physically separable solid to which a member of a binding pair can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, a solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support can be a collection of particles. In some embodiments, the particles can comprise silica, and the silica may comprise silica dioxide. In some embodiments, the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). A member of a binding pair may be linked to a solid support by covalent bonds or by non-covalent interactions and may be linked to a solid support directly or indirectly (e.g., via an intermediary agent such as a spacer molecule or biotin).

Any suitable method for carrying out proximity ligation may be used. For example, a HiC method typically includes the following steps: (1) digestion of chromatin with a restriction endonuclease (or fragmentation); (2) labelling the digested ends by filling in the 5′-overhangs with biotinylated nucleotides; and (3) ligating the spatially proximal digested ends, thus preserving spatial-proximity relationships. Once spatial-proximity relationships are preserved, further steps in a HiC method may include: purifying and enriching biotin-labelled ligation junction fragments, preparing a library from the enriched fragments and sequencing the library. In some embodiments, the biotin can be replaced with any junction marker. The term “junction marker” as used herein, refers to any compound or chemical moiety that is capable of being incorporated within a nucleic acid and can provide a basis for selective purification. For example, a junction marker may include, but not be limited to, a labeled nucleotide linker, a labeled and/or modified nucleotide, nick translation, primer linkers, or tagged linkers. The term “labeled nucleotide linker” as used herein, refers to a type of junction marker comprising any nucleic acid sequence comprising a label that may be incorporated (i.e., for example, ligated) into another nucleic acid sequence. For example, the label may serve to selectively purify the nucleic acid sequence (i.e., for example, by affinity chromatography). Such a label may include, but is not limited to, a biotin label, a histidine label (i.e., 6His), or a FLAG label. Another example of a proximity ligation method may include the following steps: (1) digestion of chromatin with a restriction endonuclease (or fragmentation); (2) blunting the digested or fragmented ends or omission of the blunting procedure; and (3) ligating the spatially proximal ends, thus preserving spatial-proximity relationships. Once spatial-proximity relationships are preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see e.g., International Patent Application Publication No. WO 2014/168575). A capture probe generally comprises a short sequence of nucleotides or oligonucleotide (e.g., 10-500 bases in length) capable of hybridizing to another nucleotide sequence. In some embodiments, a capture probe comprises a label (e.g., a label for selectively purifying specific nucleic acid sequences of interest). Labels may include, for example, a biotin or digoxigenin label. In some embodiments, capture probes are designed according to a panel of sequences and/or genes of interest.

### Crosslinking

Methods herein may include contacting a population of cells and/or cell nuclei with one or more crosslinking agents. Crosslinking generally refers to bonding one polymer to another polymer. These bonds may be covalent bonds or ionic bonds. In some applications, crosslinking is used to link DNA within a chromatin complex containing DNA and/or one or more proteins (e.g., histones) to maintain the structure of chromatin complexes. In some applications, crosslinking is used to link proteins with other proteins or polymers (e.g., membrane proteins with other membrane polymers, binding agents, or ligands). Crosslinking may include chemical crosslinking and/or UV crosslinking. Chemical crosslinking may be performed using suitable chemical crosslinking agents such as an aldehyde (e.g., formaldehyde, glutaraldehyde), disuccinimidyl glutarate (DSG), methanol, ethylene glycol bis(succinimidyl succinate) (EGS), bissulfosuccinimidyl suberate (BS3), 1-Ethyl-3-[3-dimethylaminopropyl]carbodiimide (EDC), formalin, psoralen, aminomethyltrioxsalen, mitomycin C, nitrogen mustard, melphalan, 1,3-butadiene diepoxide, cis diaminedichloroplatinum (II), cyclophosphamide, and the like and combinations thereof.

In some embodiments, nucleic acids present in a cell, a cell nucleus, or a plurality of cells and/or cell nuclei are fixed in position relative to each other by chemical crosslinking, for example by contacting the cells with one or more chemical crosslinkers. This treatment locks in the spatial relationships between portions of nucleic acids in a cell. Any suitable method of fixing the nucleic acids in their positions may be used. In some embodiments, cells and/or cell nuclei are fixed, for example with a fixative, such as an aldehyde, for example formaldehyde or glutaraldehyde. In some embodiments, a sample of one or more cells and/or cell nuclei is crosslinked with a crosslinker to maintain the spatial relationships in the cells/cell nuclei. For example, a sample of cells and/or cell nuclei can be treated with a crosslinker to lock in the spatial information or relationship about the molecules in the cells and/or cell nuclei, such as the DNA and RNA in the cell and/or nucleus. In some embodiments, the relative positions of the nucleic acid can be maintained without using crosslinking agents. For example, nucleic acids may be stabilized using spermine and spermidine. In certain instances, cell nuclei may be stabilized by embedding in a polymer such as agarose. In some embodiments, a crosslinker is a reversible crosslinker. In some embodiments, a crosslinker is reversed, for example after nucleic acid fragments or other polymers are joined. In some embodiments, nucleic acids are released from a crosslinked three-dimensional matrix by treatment with an agent, such as a proteinase, that can degrade proteinaceous material from the sample, thereby releasing the end ligated nucleic acids for further analysis, such as nucleic acid sequencing. A sample may be contacted with a proteinase, such as Proteinase K. In some embodiments, cells and/or cell nuclei are contacted with a crosslinking agent to provide crosslinked cells and/or crosslinked cell nuclei. In some embodiments, cells and/or cell nuclei are contacted with a protein-nucleic acid crosslinking agent, a nucleic acid-nucleic acid crosslinking agent, a protein-protein crosslinking agent, or any combination thereof. Using this method, nucleic acids present in a sample become resistant to spatial rearrangement and the spatial information about the relative locations of nucleic acids in a cell and/or cell nucleus is maintained. In some embodiments, a crosslinker is a reversible crosslinker, such that crosslinked molecules can be easily separated in subsequent steps a method described herein. In some embodiments, a crosslinker is a non-reversible crosslinker, such that crosslinked molecules cannot be easily separated. In some embodiments, a crosslinker is light, such as UV light. In some embodiments, a cross linker is light activated.

In some embodiments, a method herein comprises contacting a population of cells and/or cell nuclei with a first crosslinking agent and a second crosslinking agent. In such embodiments, a population of double-crosslinked cells and/or double-crosslinked cell nuclei is generated. In some embodiments, a method herein comprises contacting a population of cells and/or cell nuclei with a first crosslinking agent, thereby generating a population of single-crosslinked cells and/or single-crosslinked cell nuclei. In some embodiments, a method herein comprises contacting a population of single-crosslinked cells and/or single-crosslinked cell nuclei with a second crosslinking agent, thereby generating a population of double-crosslinked cells and/or double-crosslinked cell nuclei. In some embodiments, a first crosslinking agent comprises formaldehyde or disuccinimidyl glutarate (DSG). In some embodiments, a second crosslinking agent comprises formaldehyde or disuccinimidyl glutarate (DSG). In some embodiments, a first crosslinking agent comprises formaldehyde and a second crosslinking agent comprises disuccinimidyl glutarate (DSG).

In some embodiments, a method that generates a population of double-crosslinked cells and/or double-crosslinked cell nuclei provides an improved spatial-proximity relationship assessment and/or improved proximity ligation results when compared to an assessment and/or results generated from single-crosslinked cells and/or single-crosslinked cell nuclei. For example, an increased quantification of proximity ligated chromatin contacts (PLCC) may be observed for double-crosslinked cells and/or double-crosslinked cell nuclei compared to single-crosslinked cells and/or single-crosslinked cell nuclei. In some embodiments, a PLCC per cell and/or cell nucleus is greater for double-crosslinked cells and/or double-crosslinked cell nuclei than the PLCC per cell and/or cell nucleus obtained under conditions in which single-crosslinked cells and/or single-crosslinked cell nuclei are subjected to conditions that preserve spatial-proximity relationships. In some embodiments, a percentage of PLCC is greater for double-crosslinked cells and/or double-crosslinked cell nuclei than the percentage of PLCC obtained under conditions in which single-crosslinked cells and/or single-crosslinked cell nuclei are subjected to conditions that preserve spatial-proximity relationships.

In some embodiments, a method that generates a population of double-crosslinked cells and/or double-crosslinked cell nuclei provides improved partitioning of cells and/or cell nuclei when compared to partitioning single-crosslinked cells and/or single-crosslinked cell nuclei. For example, an increased proportion of partitions containing a single cell or single nucleus may be observed for double-crosslinked cells and/or double-crosslinked cell nuclei compared to single-crosslinked cells and/or single-crosslinked cell nuclei. Accordingly, a smaller proportion of partitions comprise two or more cells and/or cell nuclei when double-crosslinked cells and/or double-crosslinked cell nuclei are partitioned compared to single-crosslinked cells and/or single-crosslinked cell nuclei.

A proportion of partitions comprising two or more cells and/or cell nuclei may be assessed according to a multiplet rate. A multiplet rate generally is the fraction of barcodes associated with partitions having two or more cells with respect to total barcodes distributed in partitions that have at least one cell. Barcodes may be associated with partitions as described herein. In certain instances, each partition is associated with a single barcode. A total number of barcodes distributed in partitions includes (i) barcodes associated with partitions having a single cell, and (ii) barcodes associated with partitions having two or more cells. In certain instances, a total number of barcodes distributed in partitions includes (i) barcodes associated with partitions having a single cell, (ii) barcodes associated with partitions having two or more cells, and (iii) barcodes associated with partitions having no cells (although barcodes that end up in partitions having no cells are not detectable via sequencing because they don't generate library molecules).

In some embodiments, a subset of partitions comprise two or more cells and/or cell nuclei. In some embodiments, a subset of partitions comprising two more cells and/or cell nuclei comprises partitions each associated with a single barcode. A multiplet rate is a fraction of barcodes associated with two or more cells and/or cell nuclei. In some embodiments, a method herein comprises determining a multiplet rate. In some embodiments, a multiplet rate for double-crosslinked cells and/or double-crosslinked cell nuclei is less than the multiplet rate obtained under conditions in which single-crosslinked cells and/or single-crosslinked cell nuclei are subjected to partitioning. In some embodiments, a multiplet rate for double-crosslinked cells and/or double-crosslinked cell nuclei is less than the multiplet rate obtained under conditions in which single-crosslinked cells and/or single-crosslinked cell nuclei are subjected to conditions that preserve spatial-proximity relationships. In some embodiments, a multiplet rate for double-crosslinked cells and/or double-crosslinked cell nuclei is less than the multiplet rate obtained under conditions in which single-crosslinked cells and/or single-crosslinked cell nuclei are subjected to conditions that preserve spatial-proximity relationships and partitioning. In some embodiments, a multiplet rate for double-crosslinked cells and/or double-crosslinked cell nuclei is less than about 20%. In some embodiments, a multiplet rate for double-crosslinked cells and/or double-crosslinked cell nuclei is less than about 15%. In some embodiments, a multiplet rate for double-crosslinked cells and/or double-crosslinked cell nuclei is less than about 10%. For example, a multiplet rate for double-crosslinked cells and/or double-crosslinked cell nuclei may be less than about 9%, 8%, 7%, 6%, 5%, 4%, 3%, 2%, or 1%. In some embodiments, a multiplet rate for double-crosslinked cells and/or double-crosslinked cell nuclei is less than about 8%.

### Accessible Chromatin

A method herein may comprise contacting a population of cells and/or cell nuclei with one or more agents that enrich for accessible chromatin. Agents that enrich for accessible chromatin generally refer to agents used in a method in which a binding agent tags accessible chromatin regions for downstream analysis, sometimes referred to as tagmentation. Agents that enrich for accessible chromatin may include agents used in a method in which a transposase enzyme tags accessible chromatin regions for downstream analysis (e.g., Assay for Transposase Accessible Chromatin (ATAC); Single Cell Assay for Transposase Accessible Chromatin (scATAC); Single Cell Assay for Transposase Accessible Chromatin with Sequencing (scATAC-seq) (10X Genomics)). Chromatin generally compacts meters of DNA into the nucleus, where a small fraction of DNA is accessible for transcription within each cell. An ATAC method probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility. In certain workflows, cells/nuclei are transposed in bulk, followed by partitioning on a microfluidic chip into nanoliter-scale gelbead-in-emulsions (GEMs) in a specialized instrument. The transposed DNA of individual nuclei are identified with a unique barcode. Libraries are generated and sequenced, and barcodes are used to associate individual reads back to individual partitions and, thereby, each individual cell.

In certain instances, agents that enrich for accessible chromatin include agents used in an antibody-mediated tagmentation method. Certain genomic regions may be targeted by using one or more antibodies that bind to one or more regions of interest. For example, open chromatin regions specifically bound by certain proteins (e.g., RNA polymerase II) may be targeted by an antibody that specifically binds to the protein bound to the chromatin (e.g., anti-RNA polymerase Il antibody) followed by a protein A/G conjugated transposase. Other non-limiting examples of chromatin-bound molecules that may be targeted by an antibody-mediated tagmentation method include histones, methyl groups, acetyl functional groups, and transcription factors.

In some embodiments, a population of cells and/or cell nuclei is contacted with one or more agents that enrich for accessible chromatin after the cells and/or cell nuclei are crosslinked or double-crosslinked. In some embodiments, a population of cells and/or cell nuclei is contacted with one or more agents that enrich for accessible chromatin after the cells and/or cell nuclei are contacted with one or more agents that preserve spatial-proximity relationships in the nucleic acid of the cells and/or cell nuclei. In some embodiments, a population of cells and/or cell nuclei is contacted with one or more agents that enrich for accessible chromatin before the cells and/or cell nuclei are partitioned.

In some embodiments, one or more agents that enrich for accessible chromatin comprise a transposase. A transposase generally refers to an enzyme that binds to the end of a transposon and catalyzes its movement to another part of the genome by a cut and paste mechanism or a replicative transposition mechanism. In some embodiments, a transposase is a Tn5 transposase. In some embodiments, one or more agents that enrich for accessible chromatin further comprise one or more oligonucleotides. An oligonucleotide generally refers to a nucleic acid (e.g., DNA, RNA) polymer. Oligonucleotides may be short in length (e.g., less than 50 bp, less than 40 bp, less than 30 bp, less than 20 bp, less than 10 bp, less than 5 bp), and may be artificially synthesized. In some embodiments, one or more agents that enrich for accessible chromatin further comprise one or more universal oligonucleotides. In some embodiments, oligonucleotides comprise sequencing primer sites or sequencing primer sequences. In some embodiments, oligonucleotides include one or more primer binding domains. A primer binding domain is a polynucleotide to which a primer (e.g., an amplification primer, a sequencing primer) can anneal. A primer binding domain typically comprises a nucleotide sequence that is complementary or substantially complementary to the nucleotide sequence of a primer (e.g., an amplification primer, a sequencing primer). In some embodiments, oligonucleotides interact with barcoded primers in a partitioning step, as described herein.

In some embodiments, one or more agents that enrich for accessible chromatin are provided in a salt solution. In some embodiments, a salt is present at a concentration whereby the signal to noise of an accessibility signal is improved compared to the signal to noise of an accessibility signal in the absence of salt or in the presence of a different concentration of salt. In some embodiments, a salt is present at a concentration of about 25 mM to about 250 mM. In some embodiments, a salt is present at a concentration of about 50 mM to about 200 mM. For example, a salt may be present at a concentration of about 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, or 250 mM. In some embodiments, the salt is a sodium salt. In some embodiments, the salt is NaCl.

### Protein Expression

Methods herein may comprise assessment of protein expression. Methods herein may comprise assessment of cell-surface protein expression. Methods herein may comprise contacting a population of cells and/or cell nuclei with a protein binding agent. In some embodiments, a method herein comprises contacting a population of cells and/or cell nuclei with a cell-surface protein binding agent. In some embodiments, a method herein comprises contacting a population of cells and/or cell nuclei with a cell-surface protein binding agent followed by crosslinking (e.g., a crosslinking process described herein).

In some embodiments, methods herein comprise assessment of non-cell-surface protein expression (e.g., cytoplasmic protein expression, secretory pathway protein expression, organelle protein expression, nuclear membrane protein expression, intranuclear protein expression, and the like). For example, assessment of intranuclear protein expression may be performed using any suitable method (e.g., intranuclear cellular indexing of transcriptomes and epitopes (inCITE-seq) as described in Chung et al., Nature Methods volume 18, 1204-1212 (2021)). Briefly, inCITE-seq enables multiplexed and quantitative intranuclear protein measurements using DNA-conjugated antibodies coupled with RNA sequencing on a droplet-based sequencing platform. To allow antibody diffusion across a nuclear membrane, nuclei are lightly fixed with formaldehyde and permeabilized, blocked under optimized conditions to minimize non-specific binding of DNA-conjugated antibodies inside the nucleus, and combined with nucleus hashing antibodies to allow multiplexing. Antibody-stained nuclei are loaded for droplet-based single nucleus RNA sequencing (snRNAseq) for simultaneous capture of antibody DNA tags and the transcriptome.

Protein binding agents may include antibodies, antibody fragments, and/or antibody derivatives, for example. In some embodiments, a protein binding agent comprises at least one immunoglobulin heavy chain variable domain and at least one immunoglobulin light chain variable domain. In some embodiments, a protein binding agent comprises two immunoglobulin heavy chain variable domains and two immunoglobulin light chain variable domains. In certain configurations, each immunoglobulin heavy chain variable domain of a protein binding agent comprises first, second, and third heavy chain complementarity determining regions (CDRs; CDRH1, CDRH2, CDRH3), and each immunoglobulin light chain variable domain of a protein binding agent comprises first, second, and third light chain CDRs (DCRL1, CDRL2, CDRL3).

In some embodiments, a protein binding agent is associated with one or more oligonucleotides. In some embodiments, a protein binding agent is conjugated to one or more oligonucleotides. In some embodiments, a protein binding agent is an antibody and the antibody is conjugated to one or more oligonucleotides (e.g., TotalSeq™ oligo-conjugated antibodies; BioLegend, San Diego, CA). In some embodiments, an oligonucleotide comprises a poly-A tail sequence. In some embodiments, an oligonucleotide comprises a barcode that can label a specific target. In some embodiments, an oligonucleotide comprises an amplification handle, which makes it compatible with certain sequencing platforms (e.g., Illumina® sequencing). In some embodiments, antibody-oligonucleotide conjugates allow for sample multiplexing and/or multiplet detection. In some embodiments, antibody-oligonucleotide conjugates are provided as a pool of antibodies capable of recognizing ubiquitously expressed cell surface proteins (sometimes referred to as a hashtag). For human samples, the hashtag reagents recognize CD298 and B2 microglobulin, and they are tagged with the same unique DNA barcodes. For mouse samples, the surface proteins are CD45 and H-2 MHC class I, which may be tagged with their own unique DNA barcodes. In some embodiments, a method herein comprises batch-barcoding or cell hashtagging where antibody-oligonucleotide conjugates are used to label every cell within a sample prior to pooling. Several samples, labeled with such constructs, are then pooled and analyzed.

### Gene Expression

Methods herein may comprise assessment of gene expression. In some embodiments, gene expression is assessed by generating cDNA from mRNA and sequencing the cDNA (sometimes referred to as RNA-seq). In some embodiments, gene expression is assessed by generating cDNA from mRNA in a single cell and/or single nucleus and sequencing the cDNA (sometimes referred to as scRNA-seq). RNA-seq and scRNA-seq methods are known in the art and may be performed using a suitable commercial kit. Generally, a workflow includes i) isolation of single cells and/or single nuclei (for scRNA-seq), ii) lysis, iii) capture of polyadenylated mRNA using poly [T]-primers, iv) conversion of poly [T]-primed mRNA to complementary DNA (cDNA) by a reverse transcriptase, v) amplification of cDNA, and vi) sequencing. In certain applications, reverse-transcription primers comprise additional nucleotide sequences, such as adapter sequences for use with certain sequencing platforms, unique molecular identifiers (UMIs) to mark a single mRNA molecule, and/or indexes to preserve information on cellular origin. In some embodiments a method herein comprises contacting cells and/or cell nuclei with an agent comprising a reverse transcriptase activity. In some embodiments, an agent comprising a reverse transcriptase activity is a reverse transcriptase.

In some embodiments, a method herein comprises generating cDNA from mRNA in partitioned cells and/or partitioned cell nuclei. In some embodiments, a method herein comprises performing a heated crosslink reversal process prior to generating cDNA from mRNA in partitioned cells and/or partitioned cell nuclei (e.g., as described in Van Phan et al., Nature Communications volume 12, Article number: 5636 (2021)). For example, after droplet generation, droplets may be heated (e.g., on a heat block at about 56° C. for about 1 hour) to reverse crosslinks, then incubated at room temperature for about 10 minutes and optionally kept on ice for at least about 5 minutes.

In some embodiments, gene expression is assessed by hybridizing probes to mRNA in a single cell and/or single nucleus and sequencing probes as means to quantify gene expression, such as the approach taken in the 10X Genomics Fixed RNA Profiling solution. Generally, a workflow includes i) fixation of cells using a fixative (e.g., paraformaldehyde), ii) hybridization of probe sets to mRNA, where pairs of probes hybridize to target mRNA molecules, iii) isolation of single cells and/or single nuclei, iv) lysis, v) RNA-templated probe pair ligation and extension using barcode primers, vi) amplification ligated probe pairs, and vii) sequencing.

### Partitioning

Methods herein may comprise partitioning cells and/or cell nuclei. Methods herein may comprise partitioning cells and/or cell nuclei into single cells and/or single cell nuclei. In some embodiments, a method herein comprises partitioning a population of cells and/or a population of cell nuclei into partitions. The partitions may comprise partitioned single cells and/or partitioned single cell nuclei. Partitioning cells into single cells and/or single cell nuclei may be useful for obtaining sequence information from a single cell and/or single cell nucleus and/or generating a single cell profile and/or single cell nucleus profile.

In some embodiments, cells and/or cell nuclei are partitioned into droplets. For example, cells and/or cell nuclei may be partitioned using a droplet microfluidics approach. Using this approach, cells/nuclei are input into a commercial droplet microfluidics system (e.g., 10X Genomics (Pleasanton, CA), Bio-Rad, (Hercules, CA), Mission Bio (South San Francisco, CA)) where reagents are delivered to barcode and amplify DNA (e.g., proximally ligated DNA; DNA enriched for accessible chromatin) from each single cell/nucleus. Each droplet may contain a unique barcode. In some embodiments, a method herein comprises contacting a population of cells and/or cell nuclei with gelbead-in-emulsion (GEM) and one or more cell-specific and/or nucleus-specific barcode oligonucleotide species. Libraries may be produced from amplified DNA molecules of each cell/nucleus. Libraries may be sequenced, and sequence reads may be examined to obtain sequence information at single cell resolution.

In some embodiments, cells and/or cell nuclei are partitioned into physical compartments. In some embodiments, cells and/or cell nuclei are partitioned into wells (e.g., wells of a microtiter plate). In some embodiments, cells and/or cell nuclei are partitioned via cell/nuclei sorting. Cells/nuclei may be sorted using a cell sorting instrument (e.g., FACS; FANS), or manually, into discrete physical compartments such as wells of a microtiter plate. DNA (e.g., proximally ligated DNA; DNA enriched for accessible chromatin) may be purified and amplified from each single cell using methods of genome amplification known in the art, such as multiple displacement amplification (MDA), or other techniques. Such an approach is analogous to Flyamer et al. Nature, 544 (7648), pp. 110-114 (2017) or Tan et al. Science, 361 (6405), pp. 924-928 (2018). Libraries may be produced from amplified DNA molecules of each cell/nucleus. Libraries may be sequenced, and sequence reads may be examined to obtain sequence information at single cell resolution.

### Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, chromatin, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

### Nucleic Acid Library

Methods herein may comprise generating one or more nucleic acid libraries. In some embodiments a single nucleic acid library is generated for a plurality of modalities described herein. For example, a single nucleic acid library may be generated for an assessment of spatial-proximity relationships and an assessment of accessible chromatin. In certain configurations, a single nucleic acid library may be generated for an assessment of spatial-proximity relationships, an assessment of accessible chromatin, and an assessment of protein expression. In certain configurations, a single nucleic acid library may be generated for an assessment of spatial-proximity relationships, an assessment of accessible chromatin, and an assessment of gene expression. In certain configurations, a single nucleic acid library may be generated for an assessment of spatial-proximity relationships, an assessment of accessible chromatin, an assessment of protein expression, and an assessment of gene expression. In some embodiments two or more nucleic acid libraries generated for a plurality of modalities described herein in any combination. For example, a first nucleic acid library may be generated for an assessment of spatial-proximity relationships and an assessment of assessment of accessible chromatin, and a second nucleic acid library may be generated for an assessment of protein expression and/or gene expression.

In some embodiments, a method herein comprises generating one or more nucleic acid libraries from one or more of partitioned cells and/or partitioned cell nuclei. In some embodiments, one or more nucleic acid libraries comprise one or more nucleic acid library molecule species chosen from i) library molecules comprising accessible chromatin fragments; ii) library molecules comprising accessible chromatin fragments and proximity ligated chromatin fragments; iii) library molecules comprising oligonucleotides representing protein expression; and iv) library molecules comprising cDNA fragments representing gene expression.

A nucleic acid library generally refers to a plurality of polynucleotide molecules (e.g., a sample of nucleic acids; nucleic acid from a single cell or single nucleus) that are prepared, assembled and/or modified for a specific process, non-limiting examples of which include immobilization on a solid phase (e.g., a solid support, a flow cell, a bead), enrichment, amplification, cloning, detection, and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component Clq, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI), a palindromic sequence, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters. In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation.

In certain embodiments, a ligation-based library preparation method is used (e.g., ILLUMINA TRUSEQ, Illumina, San Diego CA). Ligation-based library preparation methods often make use of an adapter design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, an identifier is incorporated into a nucleic acid library. An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

### Nucleic Acid Sequencing

A method herein may comprise sequencing nucleic acid, thereby generating sequence reads. In some embodiments, a method herein comprises sequencing one or more nucleic acid libraries. In some embodiments, a method herein comprises analyzing sequence reads according to a sequence read analysis. In some embodiments, a sequence read analysis comprises identifying spatial-proximity relationship information (e.g., by analyzing the sequences of nucleic acid fragments comprising ligation junctions). In some embodiments, a sequence read analysis comprises identifying accessible chromatin information (e.g., by analyzing the sequences of nucleic acid fragments enriched for accessible chromatin). In some embodiments, a sequence read analysis comprises identifying protein expression information (e.g., by analyzing oligonucleotide tags associated with protein binding agents). In some embodiments, a sequence read analysis comprises identifying gene expression information (e.g., by analyzing cDNA sequences).

In some embodiments, a sequencing process herein comprises massively parallel sequencing (i.e., nucleic acid molecules are sequenced in a massively parallel fashion, typically within a flow cell). In some embodiments, a sequencing process herein is a shotgun sequencing process. In some embodiments, a sequencing process herein is a locus-specific sequencing process. In some embodiments, a sequencing process herein is a targeted sequencing process. In some embodiments, a sequencing process herein is a non-locus-specific sequencing process. In some embodiments, a sequencing process herein is a non-targeted sequencing process. In some embodiments, a sequencing process herein comprises single-end sequencing. In some embodiments, a sequencing process herein comprises paired-end sequencing.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), lon Torrent™ (e.g., lon PGM™ and/or lon Proton™ sequencing systems); Pacific Biosciences (e.g., PACBIO RS Il sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a sequencing process generates a plurality of sequence reads. The plurality of sequence reads may be further processed (e.g., mapped, quantified, normalized). In some embodiments, hundreds, thousands, tens of thousands, hundreds of thousands, millions, tens of millions, hundreds of millions, or billions of sequence reads are generated by a sequencing process described herein. In some embodiments, a sequencing process generates thousands of sequence reads. In some embodiments, a sequencing process generates millions of sequence reads. In some embodiments, a sequencing process generates thousands to millions of sequence reads. In some embodiments, a sequencing process generates between about 100,000 reads to about 1 billion reads. In some embodiments, a sequencing process generates between about 500,000 reads to about 100 million reads. In some embodiments, a sequencing process generates between about 1 million reads to about 10 million reads. For example, a sequencing process may generate about 1 million reads, about 2 million reads, about 3 million reads, about 4 million reads, about 5 million reads, about 6 million reads, about 7 million reads, about 8 million reads, about 9 million reads, about 10 million reads. In some embodiments, a sequencing process generates about 100,000 or more reads. In some embodiments, a sequencing process generates about 500,000 or more reads. In some embodiments, a sequencing process generates about 1 million or more reads. In some embodiments, a sequencing process generates about 5 million or more reads. In some embodiments, a sequencing process generates about 10 million or more reads.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1—, 2—, 3—, 4—, 5—, 6—, 7—, 8—, 9—, 10-, 15-, 20-, 30-, 40-, 50-, 60-, 70-, 80-, 90-fold or greater coverage). In some embodiments, a sequencing process is performed at about 0.01-fold coverage to about 1-fold coverage. In some embodiments, a sequencing process is performed at about 0.02-fold coverage. In some embodiments, a sequencing process is performed at about 0.05-fold coverage. In some embodiments, a sequencing process is performed at about 0.1-fold coverage. In some embodiments, a sequencing process is performed at about 1-fold coverage to about 30-fold coverage. In some embodiments, a sequencing process is performed at about 5-fold coverage. In some embodiments, a sequencing process is performed at a coverage of at least about 0.01-fold. In some embodiments, a sequencing process is performed at a coverage of at least about 0.1-fold. In some embodiments, a sequencing process is performed at a coverage of at least about 1-fold. In some embodiments, a sequencing process is performed at a coverage of about 0.01-fold or less. In some embodiments, a sequencing process is performed at a coverage of about 0.1-fold or less. In some embodiments, a sequencing process is performed at a coverage of about 1-fold or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, one nucleic acid sample from one cell is sequenced. In certain embodiments, nucleic acids from each of two or more cells are sequenced. In certain embodiments, nucleic acid samples from two or more cells are pooled, and the pool is sequenced. In the latter embodiments, a nucleic acid sample from each cell may be identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and Phix control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego CA)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), Singular Genomics (e.g., G4 sequencing platform), Element Biosciences (e.g., AVITI™ System), Ultima Genomics (e.g., UG 100™ sequencing platform), SOLID, Roche/454, PACBIO and/or SMRT, Helicos True Single Molecule Sequencing, lon Torrent and lon semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads) is processed prior to, or in conjunction with, an analysis of the sequenced nucleic acid. For example, sequence reads may be processed according to one or more of the following: aligning, mapping, filtering, counting, normalizing, weighting, generating a profile, and the like, and combinations thereof. Certain processing steps may be performed in any order and certain processing steps may be repeated.

### Quantitative Benchmarks

Non-limiting examples of performance assessment metrics and ranges of quantitative benchmarks (QB) for each modality (spatial proximity relationships (scHiC), accessible chromatin (scATAC), gene expression (scRNA), and protein expression (scProtein)) are described in Table1 below.

TABLE 1
Quantitative benchmarks

| Modality | Metrics (target benchmark) |
|---|---|
| scHiC (metrics measure proximally | PLCC per cell |
| ligated chromatin contacts) | (e.g., >1,000, >5,000, >10,000) |
| | % PLCC readouts |
| | (e.g., >5%, >10%, >20%, >30%, >40%, >50%) |
| scATAC (metrics measure the signal | TSS enrichment (e.g., >5, >7, >10) |
| to noise in chromatin accessibility | Fraction of reads in peaks |
| preservation and enrichment) | (e.g.., >5%, >10%, >20%, >30%) |
| | Nucleosome signal |
| | (e.g., <2, <3, <4, <5, <6) |
| scRNA (metrics measure assay sensitivity | Median UMIs per cell |
| to transcripts (UMIs) and genes per cell, | (e.g., >1000, >1250, >1500, >1750, >2000) |
| and mitochondrial content to detect dying | Median genes per cell |
| cells; targets depend on thresholds set | (e.g., >600, >700, >750, >800, >900) |
| empirically based on empirical data QC | Mitochondrial ratio |
| and generally based on prior publications) | (e.g., <0.1%, <0.2%, <0.3%, <0.4%, <0.5%) |
| scProtein (metrics assess the specificity | % hashtag ADTs correctly assigned |
| and accuracy of oligo-conjugated antibody | (e.g., >80%, >85%, >90%, >95%) |
| binding and is based on CITE-seq) | % concordance with FACS |
| | (e.g., >80%, >85%, >90%, >95%) |
| Universal (metrics include observed | Multiplet rate |
| multiplet rates, which is the expected | (e.g., <6%, <7%, <8%, <9%, <10%) |
| rate based on Poisson modeling when | Replicate concordance |
| targeting 10,000 cells per experiment | (e.g., >70%, >75%, >80%, >85%, >90%) |
| (10XG); replicate concordance; | Published data concordance |
| concordance with published data; | (e.g., >60%, >65%, >70%, >75%, >80%) |
| and concordance in the cell types | Cell types ID'd concordance |
| identified between replicates | (e.g., 90%, 95%, 100%) |
| and published data from that sample) | |

### Kits

Provided in certain embodiments are kits. The kits may include any components and compositions described herein (e.g., one or more agents that preserve spatial-proximity relationships in nucleic acid of cells and/or cell nuclei, one or more reagents that generate proximity ligated nucleic acid molecules, one or more agents that enrich for accessible chromatin, one or more agents for capturing protein expression, one or more agents for capturing gene expression, one or more agents for partitioning a population of cells and/or cell nuclei into partitions, one or more agents for generating one or more nucleic acid libraries) useful for performing any of the methods described herein, in any suitable combination. Kits may further include any reagents, buffers, or other components useful for carrying out any of the methods described herein. For example, a kit may include one or more of a first crosslinking agent, a second crosslinking agent, a DNA ligase, a reverse transcriptase, a polymerase, one or more oligonucleotides, a ligase (e.g., T4 DNA ligase), a transposase (e.g., Tn5 transposase), one or more endonucleases, one or more labeled nucleotides, a salt solution (e.g., 50 mM to 200 mM NaCl solution), one or more cell surface binding agents (e.g., antibodies conjugated to an oligonucleotide), gelbead-in-emulsion (GEM), one or more cell-specific and/or nucleus-specific barcode oligonucleotide species, and any combination thereof.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for preserving spatial-proximity relationships in nucleic acid of cells and/or cell nuclei, generating proximity ligated nucleic acid molecules, enriching for accessible chromatin, capturing protein expression, capturing gene expression, partitioning a population of cells and/or cell nuclei into partitions, and/or generating one or more nucleic acid libraries. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### Certain Implementations

Following are non-limiting examples of certain implementations of the technology.
A1. A method for preparing nucleic acid from single cells and/or single cell nuclei, comprising:
   a) contacting a population of cells and/or cell nuclei with a first crosslinking agent and a second crosslinking agent, thereby generating a population of double-crosslinked cells and/or double-crosslinked cell nuclei;
   b) contacting the population of cells and/or cell nuclei comprising double-crosslinked cells and/or double-crosslinked cell nuclei with one or more agents that preserve spatial-proximity relationships in the nucleic acid of the cells and/or cell nuclei; and
   c) partitioning the population of cells and/or cell nuclei into partitions comprising partitioned single cells and/or partitioned single cell nuclei.
A2. The method of embodiment A1, wherein (a) comprises:
   (i) contacting the population of cells and/or cell nuclei with the first crosslinking agent, thereby generating a population of single-crosslinked cells and/or single-crosslinked cell nuclei;
   (ii) contacting the population of single-crosslinked cells and/or single-crosslinked cell nuclei with the second crosslinking agent, thereby generating a population of double-crosslinked cells and/or double-crosslinked cell nuclei.
A3. The method of embodiment A1 or A2, wherein the first crosslinking agent comprises formaldehyde or disuccinimidyl glutarate (DSG).
A4. The method of any one of embodiments A1-A3, wherein the second crosslinking agent comprises formaldehyde or disuccinimidyl glutarate (DSG).
A5. The method of any one of embodiments A1-A4, wherein the first crosslinking agent comprises formaldehyde and the second crosslinking agent comprises disuccinimidyl glutarate (DSG).
A6. The method of any one of embodiments A1-A5, wherein (b) comprises contacting the population of cells and/or cell nuclei with one or more reagents that generate proximity ligated nucleic acid molecules.
A7. The method of embodiment A6, wherein (b) comprises contacting the population of cells and/or cell nuclei with one or more restriction endonucleases.
A8. The method of embodiment A7, wherein the one or more restriction endonucleases yield fragment sizes that average about 800 base pairs.
A9. The method of embodiment A7 or A8, wherein (b) comprises contacting the population of cells and/or cell nuclei with one or more restriction endonucleases for more than 2 hours.
A10. The method of embodiment A7 or A8, wherein (b) comprises contacting the population of cells and/or cell nuclei with one or more restriction endonucleases for more than 8 hours.
A11. The method of any one of embodiments A6-A10, wherein (b) comprises contacting the population of cells and/or cell nuclei with two or more restriction endonucleases.
A12. The method of any one of embodiments A6-A11, wherein (b) comprises contacting the population of cells and/or cell nuclei with a ligase.
A13. The method of any one of embodiments A1-A12, further comprising after (b) and prior to (c), contacting the population of cells and/or cell nuclei with one or more agents that enrich for accessible chromatin.
A14. The method of embodiment A13, wherein the one or more agents that enrich for accessible chromatin comprise a transposase.
A15. The method of embodiment A14, wherein the transposase is a Tn5 transposase.
A15.1 The method of embodiment A14 or A15, wherein the one or more agents that enrich for accessible chromatin further comprise one or more oligonucleotides.
A15.2 The method of embodiment A15.1, wherein the one or more oligonucleotides comprise sequencing primer sequences.
A16. The method of any one of embodiments A13-A15.2, wherein the one or more agents that enrich for accessible chromatin are in a salt solution.
A17. The method of embodiment A16, wherein the salt is present at a concentration of about 50 mM to about 200 mM.
A18. The method of embodiment A16 or A17, wherein the salt is NaCl.
A19. The method of any one of embodiments A1-A18, wherein the method further comprises prior to (a), contacting the population of cells and/or cell nuclei with a cell-surface protein binding agent.
A20. The method of embodiment A19, wherein the binding agent comprises one or more antibodies.
A21. The method of embodiment A20, wherein one or more of the antibodies are conjugated to an oligonucleotide.
A22. The method of any one of embodiments A1-A21, wherein the cells and/or cell nuclei are partitioned into droplets in (c).
A22.1 The method of embodiment A22, wherein (c) comprises contacting the population of cells and/or cell nuclei with gelbead-in-emulsion (GEM) and one or more cell-specific and/or nucleus-specific barcode oligonucleotide species.
A23. The method of any one of embodiments A1-A21, wherein the cells and/or cell nuclei are partitioned into wells in (c).
A24. The method of any one of embodiments A1-A23, further comprising after (c), performing a heated crosslink reversal process.
A25. The method of embodiment A24, further comprising after the heated crosslink reversal process, contacting cells and/or cell nuclei in the one or more partitions with an agent comprising a reverse transcriptase activity, thereby generating cDNA from mRNA present in the cells and/or cell nuclei in the one or more partitions.
A26. The method of any one of embodiments A1-A25, further comprising after (c) generating one or more nucleic acid libraries from one or more of the cells and/or cell nuclei in the partitions.
A27. The method of embodiment A26, wherein the one or more nucleic acid libraries comprise one or more nucleic acid library molecule species chosen from:
   i) library molecules comprising accessible chromatin fragments;
   ii) library molecules comprising accessible chromatin fragments and proximity ligated chromatin fragments;
   iii) library molecules comprising oligonucleotides representing protein expression; and
   iv) library molecules comprising cDNA fragments representing gene expression.
A28. The method of embodiment A26 or A27, further comprising sequencing the one or more nucleic acid libraries, thereby generating sequence reads.
A29. The method of embodiment A28, further comprising analyzing the sequence reads according to a sequence read analysis.
A30. The method of embodiment A29, wherein the sequence read analysis comprises identifying spatial-proximity relationship information.
A31. The method of embodiment A29 or A30, wherein the sequence read analysis comprises identifying accessible chromatin information.
A32. The method of any one of embodiments A29-A31, wherein the sequence read analysis comprises identifying protein expression information.
A33. The method of any one of embodiments A29-A32, wherein the sequence read analysis comprises identifying gene expression information.
A34. The method of any one of embodiments A1-A33, wherein cells and/or cell nuclei in the partitions comprise proximally ligated chromatin contacts (PLCC).
A35. The method of embodiment A34, further comprising determining an amount of proximally ligated chromatin contacts (PLCC) per cell and/or cell nucleus.
A36. The method of embodiment A35, wherein the PLCC per cell and/or cell nucleus is greater than the PLCC per cell and/or cell nucleus obtained under conditions in which single-crosslinked cells and/or single-crosslinked cell nuclei are subjected to conditions that preserve spatial-proximity relationships.
A37. The method of embodiment A35 or A36, wherein the PLCC per cell and/or cell nucleus is about 5,000 or greater.
A38. The method of any one of embodiments A34-A37, further comprising determining a percentage of proximally ligated chromatin contacts (PLCC).
A39. The method of embodiment A38, wherein the percentage of PLCC is greater than the percentage of PLCC obtained under conditions in which single-crosslinked cells and/or single-crosslinked cell nuclei are subjected to conditions that preserve spatial-proximity relationships.
A40. The method of any one of embodiments A1-A39, wherein a subset of partitions comprise two or more cells and/or cell nuclei.
A41. The method of embodiment A40, wherein:
the subset of partitions comprising two more cells and/or cell nuclei comprises partitions each associated with a single barcode; and
a multiplet rate is a fraction of barcodes associated with two or more cells and/or cell nuclei.
A42. The method of embodiment A41, further comprising determining the multiplet rate.
A43 The method of embodiment A41 or A42, wherein the multiplet rate is less than the multiplet rate obtained under conditions in which single-crosslinked cells and/or single-crosslinked cell nuclei are subjected to conditions that preserve spatial-proximity relationships.
A44. The method of any one of embodiments A41-A43, wherein the multiplet rate is less than about 8%
B1. A kit comprising:
   a) a first crosslinking agent;
   b) a second crosslinking agent; and
   c) one or more agents that preserve spatial-proximity relationships in nucleic acid of cells and/or cell nuclei.
B1.1 The kit of embodiment B1, further comprising one or more agents for partitioning a population of cells and/or cell nuclei into partitions comprising partitioned single cells and/or partitioned single cell nuclei.
B2. The kit of embodiment B1 or B1.1, wherein the first crosslinking agent comprises formaldehyde or disuccinimidyl glutarate (DSG).
B3. The kit of any one of embodiments B1-B2, wherein the second crosslinking agent comprises formaldehyde or disuccinimidyl glutarate (DSG).
B4. The kit of any one of embodiments B1-B3, wherein the first crosslinking agent comprises formaldehyde and the second crosslinking agent comprises disuccinimidyl glutarate (DSG).
B5. The kit of any one of embodiments B1-B4, wherein the one or more agents that preserve spatial-proximity relationships in the nucleic acid of the cells and/or cell nuclei comprise one or more reagents that generate proximity ligated nucleic acid molecules.
B6. The kit of embodiment B5, wherein the one or more agents in (c) comprise one or more restriction endonucleases.
B7. The kit of embodiment B5, wherein the one or more agents in (c) comprise two or more restriction endonucleases.
B8. The kit of any one of embodiments B5-B7, wherein the one or more agents in (c) comprise a ligase.
B9. The kit of any one of embodiments B1-B8, further comprising one or more agents that enrich for accessible chromatin.
B10. The kit of embodiment B9, wherein the one or more agents that enrich for accessible chromatin comprise a transposase.
B11. The kit of embodiment B10, wherein the transposase is a Tn5 transposase.
B12. The kit of embodiment B10 or B11, wherein the one or more agents that enrich for accessible chromatin further comprise one or more oligonucleotides.
B13. The kit of embodiment B12, wherein the one or more oligonucleotides comprise sequencing primer sequences.
B14. The kit of any one of embodiments B9-B14, wherein the one or more agents that enrich for accessible chromatin are in a salt solution.
B15. The kit of embodiment B14, wherein the salt is present at a concentration of about 50 mM to about 200 mM.
B16. The kit of embodiment B14 or B15, wherein the salt is NaCl.
B17. The kit of any one of embodiments B1-B16, further comprising a cell-surface protein binding agent.
B18. The kit of embodiment B17, wherein the binding agent comprises one or more antibodies. B19. The kit of embodiment B18, one or more of the antibodies are conjugated to an oligonucleotide.
B20. The kit of any one of embodiments B1.1-B19, wherein the one or more agents for partitioning a population of cells and/or cell nuclei into partitions comprise gelbead-in-emulsion (GEM) and one or more cell-specific and/or nucleus-specific barcode oligonucleotide species.
B21. The kit of any one of embodiments B1-B20, further comprising an agent comprising a reverse transcriptase activity.
B22. The kit of any one of embodiments B1-B21, further comprising one or more agents for generating one or more nucleic acid libraries.

### EXAMPLES

The examples set forth below illustrate certain implementations and do not limit the technology.

### Example 1: Development of Scalable Sc3DGR Chemistry for scHiC, scATAC, scRNA, and scProtein

In this Example, the development of scalable single-cell 3D gene regulation (sc3DGR) chemistry for scHiC, scATAC, scRNA and scProtein is described. In one aspect, a droplet-based scHiC workflow was developed that is upstream of and integrates with 10XG Chromium. Additionally, capabilities were developed for multiomic analysis of scHiC, scATAC, scRNA and scProtein from single-cells (FIG. 1). In certain sc3DGR workflows described herein, scHiC was analyzed in a multiomic context, and its measurement was dependent on the scATAC modality (FIG. 1)—i.e., the chromatin interactions captured from sc3DGR were only those associated with the accessible regulatory regions defined by scATAC.

sc3DGR chemistry optimizations described herein were performed on commercial mouse and human cell lines (from Coriell, ATCC). Workflows described herein may be performed using a sc3DGR kit. For example, a sc3DGR kit when combined with a 10XG ATAC kit captures scHiC and scATAC (optional: scProtein), or a sc3DGR kit when combined with 10XG Multiome kit captures scHiC, scATAC and scRNA.

### Sc3DGR Chemistry for Concurrent ScHiC and ScATAC

To enable scHiC on 10XG, a transposase (Tn5, a reagent used in scATAC) was used to insert into chromatin of cells that are proximity ligated (FIG. 1, Step 1e) to enable GEM barcoding, NGS, and analysis of chromatin interactions (FIG. 1). Because Tn5 inserts into accessible chromatin, and because accessible chromatin mark regulatory regions, chromatin interactions detected here originate from such regulatory regions-interactions that inform gene regulation. This approach enriches for regions of interest and saves NGS-cost.

As initial proof of concept (PoC), sc3DGR chemistry was performed on a mix of 2 mouse leukemia cell lines (RN2 and B-ALL) and 2 human leukemia cell lines (MOLM13 and NALM6). Because scHiC analyses require chromatin crosslinking, 1% formaldehyde was used. Digestion and ligation efficiency was assessed (FIG. 2, panel A). The sc3DGR workflow involves considerable cell manipulations prior to tagmentation & droplet barcoding (FIG. 1)-manipulations that are not performed in traditional scATAC-Seq (FIG. 1). Accordingly, the condition of the cells leading to 10XG (FIG. 1, Step 1a-1d) was photo-documented using light microscopy. The photo-documentation revealed substantial cell clumping after proximity ligation (FIG. 2, panel B), and NGS revealed a high (55%) multiplet rate, >10-fold higher than control scATAC-Seq (FIG. 2, panels C and D). High multiplet rates confound single-cell analyses. The biochemical steps leading up to 10XG were manipulated and the impact on cell clumping was assessed. Sequentially crosslinking the cells with formaldehyde and DSG prior to sc3DGR eliminated clumping and still enabled efficient scHiC chemistry (FIG. 2, panels E and F). Without being limited by theory, stronger crosslinking may have rendered cells resilient to manipulations (e.g., detergent treatments) that would otherwise cause rupture and clumping. Deep NGS was performed and an industry-acceptable<5% multiplet rate was observed (FIG. 2, panel G).

Next, baseline performance of the scHiC and scATAC modalities in sc3DGR data from human/mouse cell mixture was established (FIG. 3). For the scATAC modality within sc3DGR, similar accessibility signals at genes (FIG. 3, panel A), genome-wide via TSS enrichment (FIG. 3, panel B), and % FRIPs (FIG. 3, panel C) compared to control scATAC-seq were observed. Further, clustering analysis of the scATAC modality could delineate the two leukemia cell lines (FIG. 3, panels D and E). For the scHiC modality within sc3DGR, the PLCC per cell was 5K (FIG. 3, panel G), and % PLCC readouts was 6% (FIG. 3, panel F). Finally, multiomic sc3DGR analyses identified known accessibility patterns and 3D neighborhoods at MYC in MOLM13 cells (FIG. 3, panel H) and resolved cell-type specific accessibility and chromatin interactions unique to MOLM13 (Fia. 3. panel I). This indicates that the sc3DGR data is useful to reveal mechanistic gene level 3DGR landscapes.

For scATAC modality, signal: noise of accessibility signal (FIG. 3, panels A-E) may be improved by modifying salt concentrations in the tagmentation reaction. Shallow NGS analysis of a salt titration experiment in sc3DGR showed improvements (FIG. 4, panel A). Other optional modifications include tagmenting before scHiC steps, mimicking conditions of control scATAC-seq to produce high quality scATAC modality within sc3DGR. For the scHiC modality, % PLCC readouts may be improved by modifying chromatin digestion via longer digests, more restriction enzymes, and/or more units of restriction enzymes, optionally in the context of the tagmentation modifications described above. FIG. 4, panel B, shows extending the dual restriction enzyme digest to overnight digestion doubled the % PLCC readouts for sc3DGR from 6% to 13% based on shallow NGS, indicating viability for improving the scHiC modality.

Expanding Sc3DGR to scProtein Modality

Expanding sc3DGR to include cell surface protein expression (scProtein) is useful for studying immunology and immunooncology, for example. scProtein tech uses antibodies (Ab) conjugated to oligonucleotides to “stain” (i.e., bind to) cell surface proteins, which are barcoded via 10XG scATAC reagent kits and sequenced to quantify protein abundance on each cell (FIG. 1). scProtein may be useful for deep immune cell “phenotyping” of the tumor-specific immune compartment, for example. In sc3DGR, a scProtein modality not only informs immune cell composition, but enables detailed sc3DGR analyses of immune cells to shed light on how their genes are regulated in tumor environments. In addition, scProtein may be used for “hashtagging,” where antibody-conjugated oligonucleotides bind “universal” cell surface proteins (e.g., CD298), and may be leveraged for increasing sample throughput/plexity. For proof of concept, FACS was used to optimize antibody staining. Conditions were tested where cells were crosslinked, stained via hashtag Ab conjugated to a fluorophore (FITC), subjected to scHiC steps (FIG. 1, step 1), and analyzed via FACS. As a control, same was performed but without scHiC steps. Surprisingly, essentially no FITC signal (i.e., no Ab staining) was observed when scHiC steps were included (FIG. 5, panels A and B), indicating that Abs were unable to remain bound to proteins due to HiC specific detergent treatments. To address this issue, cells were stained with Abs and then crosslinked to “fix” the antibodies to their target proteins, and the successful results are shown in FIG. 5, panel C. For proof of concept of the entire sc3DGR workflow (FIG. 1), two human leukemia cell lines and two mouse leukemia cell lines each were stained with their own unique hashtags, the cells were mixed together, crosslinked, and sc3DGR (scHiC, scATAC, scProtein) was carried out with deep NGS.>95% of hashtags were appropriately derived from their respectively stained cell line (FIG. 5, panels D and E), indicating specific scProtein modality. These findings may be validated via hashtagging in PBMCs and solid tissues, small immunophenotyping Ab panels (e.g., CD10, CD33) in cell lines, and high-plex Ab panels in PBMCs and tissues. Validations may be preceded via FACS validation, followed by sc3DGR validation of all three modalities (scHiC, scATAC, scProtein) with controls.

Sc3DGR Chemistry with ScRNA Modality

Single-cell multiomic profiling of scHiC, scATAC, and scRNA may be useful for understanding diverse cellular function. Commercial assays (10XG Multiome Kit) and derivatives built on 10XG may be used to integrate scATAC+scRNA, and sc3DGR adds the scHiC modality without compromising the others. While scRNA-seq on formaldehyde fixed cells is not currently routine due to concern that fixation inhibits mRNA capture and reverse transcription (RT), high quality scRNA-seq with droplet barcoding using formaldehyde fixed cells may be achieved if the workflow includes a 1 hr heated crosslink reversal step after droplet encapsulation but before RT (FIG. 1, between Step 2 and 3). The scRNA modality is incorporated by modifying sc3DGR workflow in two ways: (i) incorporating a modified crosslink reversal protocol in the context of the sc3DGR workflow; and (ii) using 10XG Multiome kits that have been designed for tagmentation and droplet barcoding of accessible chromatin and RNA (FIG. 1, Steps 1e, 2-4). The 10X Multiome chemistry allows for an in-droplet crosslink reversal step prior to RT. Experiments are carried out in human/mouse mixtures, and then further developed and validated in PMBCs and brain tissue and with controls. Another option is to incorporate scISSACC-seq for analyzing scATAC and scRNA modalities into Sc3DGR. scISSACC-seq is advantageous in that Tn5 can tagment DNA/RNA hybrids after RT, and scISSACCseq provides equivalent gene expression analyses as traditional scRNA-seq. If scISSACC-seq steps are incorporated prior to schiC steps, the need for RT on fixed cells is eliminated 10XG scATAC kits may be used (instead of Multiome).

### Sc3DGR Bioinfo Tools

Development of new sc3DGR data requires development new bioinformatics workflows. One platform for singe-cell analysis is open source Bioinfo. Open-source tools for scATAC, scRNA, and scProtein analyses (e.g., Seurat, Signac) may be built upon and integrated with scHiC-specific tools and functionalities (FIG. 6). Based on a preliminary bioinformatics understanding, a bioinformatics workflow may be developed where scHiC and scATAC modalities are aligned with BWA-mem and sorted with Samtools. scATAC-specific pre-processing analyses (e.g., QC, peak calling, cell x accessibility matrices) are performed using Sinto, and downstream analyses (e.g., clustering, differential peak calling, TF motifs, data visualization) are carried out via Signac. scHiC-specific analyses include creating interaction maps via GenomicRanges/InteractionSet, which feeds into Sianac for downstream integrative multiomic analyses, such as multiomic clustering. For scProtein or scRNA, Kallisto/Bustools are used for alignment, QC, quantifying gene and antibody derived tag (ADT) counts, and creating cell x gene or cell x ADT matrices, respectively. The output flows into Seurat for downstream analyses (e.g., multiomic clustering, differential expression, data visualization). Once cell types are identified through clustering, statistically significant chromatin interactions are called using MAPS, which analyzes chromatin interactions derived from regulatory regions. Workflows may be containerized using Docker and Singularity.

### Materials and methods

The following materials and methods were used to perform certain assays described above.

The following protocol is for a sc3DGR approach combining a 3C method with 10× Chromium ScATAC. The workflow described below does not include biotinylation steps and targeted tagmentation. One or both features may be included in other workflow configurations. The purpose of the sc3DGR workflow is to generate high quality chromatin accessibility profiles (comparable to scATAC-seq alone) combined with high quality chromatin 3D architecture information. Results showed that sc3DGR can achieve up to 15% reads that are Hi-C contacts among the total, and can capture chromatin accessibility with some background noise. To reduce background noise and increase Hi-C contacts percentage, two parameters were tested: 1) NaCl concentration (0 mM, 25 mM, 50 mM, 100 mM, 200 mM) and 2) the number of restriction enzymes (Nlalll+Mbol, Nlalll+Mbol+Mse1). These test experiments were run with a bulk assay that uses 50,000 cells for tagmentation. Tables 2-8 provide materials used for certain aspects of the sc3DGR workflow.

TABLE 2
ADT/HTO staining buffer

| | Final | Stock | |
|---|---|---|---|
| Staining | concentration | Concentration | 20 ml |
| BSA | 2% | 10% | 4 ml |
| Tween | 0.01% | 10% | 20 μl |
| PBS | | | 16 ml |

TABLE 3
Wash buffer 10x stock

| | Working | Working | Working | Working |
|---|---|---|---|---|
| Wash Stock | Conc. | Conc. | Conc. | Conc. |
| Tris-HCl (pH 7.5) | 10 mM | 1M | 100 mM | 5 ml |
| NaCl | 10 mM | 5M | 100 mM | 1 ml |
| Tween | 0.1% | 10% | 1% | 5 ml |
| Water | | | | 39 ml |

TABLE 4
Lysis buffer 10x stock

| | Working | Reagent | 10x | 10 ml | |
|---|---|---|---|---|---|
| Lysis stock | conc. | stock conc. | stock conc. | 10x stock | |
| Tris-HCl (pH 7.5) | 10 mM | 1M | 100 mM | 1 | ml |
| NaCl | 10 mM | 5M | 100 mM | 200 | μl |
| MgCl₂ | 3 mM | 1M | 30 mM | 300 | μl |
| IGEPAL (NP40) | 0.1% | 10% | 1% | 1 | ml |
| Tween | 0.1% | 10% | 1% | 1 | ml |
| Water | | | | 6.5 | ml |

TABLE 5
Wash buffer I

| | reagent | |
|---|---|---|
| | stock conc. | 10 ml |
| wash buffer 10x stock | | 1 ml |
| bsa | 10% | 1 ml |
| water | | 8 ml |

TABLE 6
Wash buffer II (w/EDTA)

| | Reagent | | |
|---|---|---|---|
| | stock conc. | 10 ml | |
| Wash Buffer 10x Stock | | 1 | ml |
| EDTA (final: 10 mM) | 0.5M | 200 | μl |
| BSA | 10% | 1 | ml |
| Water | | 7.8 | ml |

TABLE 7
Lysis buffer

| | Final | Stock | | |
|---|---|---|---|---|
| OMNI lysis | Conc. | Conc. | 2 ml | |
| Lysis Buffer | | 10x | 200 | μl |
| 10x Stock | | | | |
| Digitonin | 0.01% | 10% | 4 | μl |
| BSA | 1% | 10% | 200 | μl |
| Water | | | 1.6 | ml |

TABLE 8
Antibodies used for cell surface protein expression

| TotalseqA | Catalog | Clone | rxn volume |
|---|---|---|---|
| CD10 | A0062 | Hl10a | 0.25 |
| CD19 | A0050 | H1B19 | 0.25 |
| CD20 | A0100 | 2H7 | 0.25 |
| CD33 | A0052 | | 0.25 |
| CD123 | A0064 | | 0.25 |
| CD11b | A0161 | ICRF44 | 0.25 |

The following protocols were used for certain aspects of the sc3DGR workflow.

### Cell Staining

1. Obtain all single cell suspensions (filter if needed) and measure viability and density (if viability is <90% it is encouraged to proceed with live cell enrichment).
2. Resuspend 1~2 million cells in 100 μl Staining buffer (2% BSA, 0.01% Tween in PBS).
3. Add 10 μl Fc Blocking reagent (FcX, BioLegend; mouse and human, respectively).
4. Incubate for 15 minutes on ice.
5. While cells are incubating in Fc Block, prepare antibody pool (panel or titrated amounts).
6. Aliquot the samples into 4 tubes (1 μl hashing antibody was added separately).
7. Incubate for 30 minutes on ice.
8. Wash cells three times with 1 ml staining buffer by centrifuging at 650 rcf for 5 min at 4° C.
9. Pool all samples together.
10. Add antibody-oligo pool to cells. (1.25 μg/ml per antibody).
11. Incubate for 30 minutes on ice.
12. Wash cells 3 times with 1 ml staining buffer by centrifuging at 650 g for 5 min at 4° C.
13. Resuspend cells in 1 ml PBS.

### Double Crosslinking

14. Add 7.5 ml PBS, 1 ml FBS, and 500 μl Formaldehyde (typically 5 million cells were fixed with 1% Formaldehyde in 10 ml buffer).
15. Rotation for 10 min RT.
16. Add 500 μl 2.5 M Glycine to quench Formaldehyde crosslinking and rotate for 5 min RT (final concentration 125 mM).
17. Centrifuge at 650 rcf for 5 min.
18. Resuspend the cells in 1 ml PBS (1% BSA) and centrifuge at 650 rcf for 5 min.
19. Resuspend the cells in 900 μl PBS.
20. Add 100 μl 30 mM DSG for further crosslinking.
   Note that DSG should be diluted to 30 mM in DMSO (do not use 300 mM stock directly).
   300 mM DSG stock solution in DMSO (RT-50 mg, add 510.84 μl DMSO for 300 mM).
21. 40 min with rotation at RT.
22. Add 160 μl 2.5M Glycine and rotate for 5 min to quench the crosslinking (final concentration: 0.4 M glycine).
23. Resuspend the cells in 1 ml PBS (1% BSA) and centrifuge at 650 rcf for 5 min.
24. (Optional) Filter the cell using Flowmi Cell Strainer. Lysis
25. Resuspend in 100 μl (per million cells) chilled lysis buffer, pipette mix 10× and incubate for 10 min on ice (5 min for primary cells).
26. Add 1 ml Wash Buffer I to the lysed cells. Pipette mix 5x.
27. Centrifuge at 650 rcf for 5 min at 4° C.
28. Resuspend in 45 μl of 1x CutSmart buffer. For regular ATAC-seq, pellet cells and discard supernatant and resuspend in 100 μl of 10x Genomics' Nuclei buffer (1X) and store at 4° C. until proximity ligation is done.

### Conditioning

29. Add 45 μl of 1% SDS (final concentration: 0.5% SDS).
   Don't pipette mix or vortex-instead gently tap or invert the tube a few times to mix it.
   Some cells may be sensitive and damaged (ruptured) by 0.5% SDS; if that is the case, use 0.25% SDS but note that this could reduce digestion efficiency.
30. Incubate tubes at 62° C. for 10 minutes in a PCR machine.
31. Add 10 μl of 10% Triton X-100 to SDS treated samples and incubate at 37° C. for 10 min. Don't pipette mix or vortex-instead gently tap or invert the tube a few times to mix it.
32. Add 100 μl Wash Buffer Il and incubate 1 min on ice.
33. Centrifuge at 350 rcf for 5 min at 4° C.
   This step is a critical step that could cause cell aggregates. Use 350 rcf for the first two centrifugations, but not over 600 rcf.
34. Remove 180 μl of supernatant and add 200 μl Wash Buffer I.
35. Centrifuge at 350 rcf for 5 min at 4° C.
36. 180 μl of supernatant and add 200 μl Wash Buffer I.
37. Centrifuge at 650 rcf for 5 min at 4° C.
38. Carefully remove supernatant without disturbing the pellet.

### Digestion

39. Discard supernatant and resuspend in 50 μl of water and transfer to a new PCR tube.
40. Add 50 μl of a master mix containing the following reagents:

TABLE 9
Digestion (2 enzymes)

| | Volume per | |
|---|---|---|
| Reagent | reaction | 10% extra |
| Water | 14 | 15.4 |
| 10X CutSmart | 10 | 11 |
| Buffer (NEB) | | |
| Enzyme 1 (Nlalll) | 6 | 6.6 |
| Enzyme 2 (Mbol) | 6 | 6.6 |
| 7X Proteinase | 14 | 15.4 |
| inhibitor | | |
| cocktail | | |
| (EDTA free) | | |
| Total | 50 | |

TABLE 10
Digestion (3 enzymes)

| | Volume per | |
|---|---|---|
| Reagent | reaction | 10% extra |
| Water | 8 | 15.4 |
| 10X CutSmart | 10 | 11 |
| Buffer (NEB) | | |
| Enzyme 1 (Nlalll) | 6 | 6.6 |
| Enzyme 2 (Mbol) | 6 | 6.6 |
| Enzyme 3 (Mse1) | 6 | 6.6 |
| 7X Proteinase | 14 | 15.4 |
| inhibitor | | |
| cocktail | | |
| (EDTA free) | | |
| Total | 50 | |

41. Mix gently by pipetting, and incubate at 37° C. overnight (set the lid temperature to 42° C.). One may do interval shaking (900 rpm, 30 sec on, 4 min off) if Thermomixer is available (currentprotocals.onlinelibrary.wiley.com/doi/full/10.1002/cpz1.198), or mix by gentle pipetting 2 or 3 times, periodically.
42. Add 100 μl of Wash Buffer II.
43. Centrifuge at 650 rcf for 5 min at 4° C.
44. Carefully discard supernatant.
45. Resuspend the sample in 200 μl of Wash Buffer I.
46. Centrifuge at 650 rcf for 5 min at 4° C.
47. Carefully discard supernatant.
48. Gently add 200 μl of Wash Buffer I.
49. Centrifuge at 650 rcf for 5 min at 4° C.
50. Carefully remove supernatant without disturbing the pellet.
51. Resuspend the sample pellet in 55 μl deionized water by gently pipetting 5-10 times. Take 5 μl and set aside for Digestion QC (final volume: 50 μl).

### Ligation

52. Add 50 μl of a master mix containing the following reagents:

TABLE 11
Ligation

| | Volume per |
|---|---|
| Reagent | reaction |
| Water | 10 |
| T4 ligase buffer | 10 |
| T4 ligase (5 U/μl) | 12 |
| 50% PEG 4000 | 4 |
| (final conc. 2%) | |
| 7x Roche | 14 |
| complete, EDTA- | |
| free Protease | |
| inhibitor | |
| Total | 50 |

53. Mix gently by pipetting, and incubate at RT for 2 hours. Mix the sample solution by pipetting after 1 hour incubation, or incubate on the rotator.
54. Add 100 μl Wash buffer II.
55. Centrifuge at 650 rcf for 5 min and carefully remove supernatant.
56. Resuspend the sample in 200 μl Wash buffer I.
57. Centrifuge at 650 rcf for 5 min and carefully remove supernatant.
58. Resuspend the sample in 200 μl Wash buffer I and filter the cells using FLOWMI Cell Strainer.
59. Centrifuge at 650 rcf for 5 min and carefully remove supernatant.
60. Resuspend in 50 μl of 1X Nuclei Buffer (10X Genomics) and count the cells.

### Tagmentation (Bulk Assay)

61. Resuspend 50,000 cells in 4 μl of nuclei buffer and mix with the tagmentation reagents as follows.

TABLE 12
Tagmentation (Bulk assay)

| | 0 mM | 25 mM | 50 mM | 100 mM | 200 mM |
|---|---|---|---|---|---|
| Reagent | NaCl | NaCl | NaCl | NaCl | NaCl |
| Sample | 4 μl | 4 μl | 4 μl | 4 μl | 4 μl |
| TD buffer | 10 μl | 10 μl | 10 μl | 10 μl | 10 μl |
| TDE | 4 μl | 4 μl | 4 μl | 4 μl | 4 μl |
| NaCl (2M) | 0 μl | 0.5 μl (1M) | 0.5 μl | 1 μl | 2 μl |
| Water | 2 μl | 1.5 μl | 1.5 μl | 1 μl | 0 μl |
| Total | | | | | 20 |

### Library Construction (Bulk Assay)

Follow the original ATAC protocol

(currentprotocals.onlinelibrary.wiley.com/doi/10.1002/0471142727.mb2129s109).
62. Immediately following transposition, purify using a Qiagen MINELUTE PCR Purification Kit.
63. Elute transposed DNA in 20 μl elution buffer (Buffer EB from the MINELUTE kit comprising 10 mM Tris⊕ CI, pH 8).
64. To amplify transposed DNA fragments, combine the following in a PCR tube:
   20 μl transposed DNA
   2.5 μl of 25 UM PCR primer 1
   2.5 μl of 25 UM barcoded PCR primer 2
   25 μl NEB Next High-Fidelity 2X PCR Master Mix (New England Labs #M0541)
      if using hot start NEB PCR master mix, incubate master mix at 98° C. for 1 min. After cooling down in ice, add to samples.
65. Terminal cycle as follows:
   1) 72° C., 5 min
   2) 98° C., 30 sec
   3) 98° C., 10 sec
   4) 63° C., 30 sec
   5) 72° C., 1 min
   6) Repeat steps 3-5. 4× (total 5 cycles)
   7) Hold at 4° C.
66. For determining PCR cycles, do test PCR reaction as follows:
   5 μl 5 cycles PCR amplified DNA
   1.5 μl H₂O
   1.25 μl of 5 μM PCR primer 1 (⅕ diluted)
   1.25 μl of 5 μM PCR barcoded primer 2 (⅕ diluted)
   1 μl of 10X SYBR ( 1/10 diluted)
   5 μl of NEB PCR master mix
67. Thermal cycle as follows
   1) 98° C., 30 sec
   2) 98° C., 10 sec
   3) 63° C., 30 sec
   4) 72° C., 1 min
   5) repeat steps 2-4, 19× (total 20 cycles)
   6) Hold at 4° C.
68. Calculate the number of cycles that corresponds to ¼ of maximum fluorescent intensity.
69. Run the remaining 45 μl PCR reaction to the correct number of cycles.
   1) 98° C., 30 sec
   2) 98° C., 10 sec
   3) 63° C., 30 sec
   4) 72° C., 1 min
   5) repeat steps 2-4, x times
   6) Hold at 4° C.
70. Vortex to resuspend SPRISELECT reagent. Add 40 μl SPRISELECT reagent to each sample. Pipette mix.
71. Incubate 5 min at room temperature.
72. Place on the magnet⊕ High until the solution clears.
73. Transfer 130 μl supernatant to a new strip tube. DO NOT discard the supernatant.
74. Vortex to resuspend SPRISELECT reagent. Add 74 μl SPRISELECT reagent to each sample. Pipette mix.
75. Incubate 5 min at room temperature.
76. Place on the magnet⊕ High until the solution clears. Remove the supernatant. Add 200 μl 80% ethanol to the pellet. Wait 30 sec.
77. Remove the ethanol.
78. Repeat ethanol washing for a total of 2 washes.
79. Centrifuge briefly. Place on the magnet⊕ Low.
80. Remove remaining ethanol.
81. Remove from the magnet. Immediately add 20.5 μl Buffer EB. Pipette mix.
82. Incubate 2 min at room temperature.
83. Centrifuge briefly. Place on the magnet⊕ Low until the solution clears.
84. Transfer 20 μl sample to a new tube strip.
85. Store at 4° C. for up to 72 h or at −20° C. for long-term storage.

### Tagmentation (Single-Cell Assay)

86. Proceed according to 10× with the below modifications:
   1) During the barcoding reaction (step 2.1) spike in 0.5 μl of 1 UM bridge oligo (there is no dead volume in the reaction, so final volume will be 65.5 μl for v1 and 60.5 μl for v1.1).
   2) During GEM incubation (step 2.5), include a 5 min incubation at 40° C. at the beginning of the protocol, i.e., 40° C. 5 min 72° C. 5 min 98° C. 30 sec 98° C. 10 sec | 59° C. 30 sec | repeat 11× (total 12 cycles) 72° C. 1 min | 15° C. hold (this extra step is not essential when using TSA products, but increases efficiency in TSB and especially TSC tag capture).
   3) During silane bead elution (step 3.10) add 43.5 μl of Elution Solution I and subsequently recover ~43 μl. Keep 3 μl aside to use as input in the tag library PCR and with the remaining 40 μl proceed to SPRI clean up as per protocol.

### Library Construction of HTO

4) During SPRI cleanup (step 3.2d), save the supernatant. For the bead bound fraction proceed as per protocol. For the sup fraction, add 32 μl SPRI, let bind for 5 min. Collect beads on magnet, wash 2× with 80% EtOH, remove remaining ethanol and elute beads in 42 μl EB (or more if multiple indexing reactions need to take place, i.e., TSA and TSB products have been used). This can be combined with the 3 μl set aside after the silane purification, as input in the ADT/HTO indexing reaction:
PCR:
   50 μl 2× KAPA mix
   2.5 μl P5 10 UM
   2.5 μl RPxx (TSA family) or D7xx 10 UM
   (D7xx (Hashtags, TotalseqB), RPxx (TotalseqA))
   3-45 μl input fragments, water to 100 μl total
   (D701 was used for this experiment. For HTO, D7 primers should be used. For 3′ ADT panel, RP primers should be used.)

### Program:

95° C. 3 min
95° C. 20 sec
60° C. 30 sec, repeat ~13-15× (total 14-16 cycles)
72° C. 20 sec
72° C. 5 min
4° C. hold
14 cycles for this experiment.
   64° C. for HTO and 60° C. for ADT (totalseq-A) Expected product: 190 base pairs (bp)
87. Perform QC through gel running (2.5 μl), qubit and bioanalyzer.

### Example 2

It was observed by Applicants that there was a relatively low percentage of chromatin interactions in the final sequencing data in certain embodiments described in Example 1 above (see, e.g., FIG. 3f). Without being bound by theory, it is believed that the inability to sequence the chromatin interaction library molecules separate from the accessible chromatin library molecules may be playing a role. In other words, because the percentage of library molecules comprising chromatin interactions is low, one would have to sequence the Drop-C library (comprising a mix of chromatin interaction fragments and accessible chromatin fragments) exceptionally deeply to obtain sufficient chromatin interactions. In doing this, lots of reads are wasted on sequencing the accessible chromatin fragments way more than what is necessary for analysis. The result of this is very high cost. Ideally, the chromatin interaction library and accessible chromatin library could be separated, so that one can sequence each library separately to its appropriate depth.

To solve for this Applicants used a biotin incorporation step after restriction digestion (and digested with restriction enzymes enabling fill-in with a biotinylated dATP) but before proximity ligation. Then after the completion of Step 3.1 in the 10X GENOMICS™ user guide, link provided below, the biotinylated chromatin interaction fragments were separated from the rest of the molecules, and then subsequent library prep steps are completed on the chromatin interaction fragments separately from the accessible chromatin fragments.

See example methods described below.

FIG. 8 shows one embodiment of a single-cell 3D gene regulation (sc3DGR) workflow. In this example, a sc3DGR workflow is carried out in bulk phase (top panel) followed by single-cell phase (bottom panel). Top panel: Step 1a includes an optional incorporation of a single-cell protein (scProtein) modality; cells are stained using oligo-conjugated antibodies (Abs). Cells are then universally crosslinked. Steps 1b-1d include chromatin conditioning, digestion, and proximity ligation. The small circle in Step 1d is biotin, which is attached to a nucleotide used during the biotin fill-in reaction. The large circle in Step 3a is a solid substrate and the Y-shaped molecule attached to the solid substrate is a streptavidin molecule. To incorporate a single-cell Transposase-Accessible Chromatin (scATAC) modality and to enable a single-cell High-throughput chromatin capture (scHiC) modality, chromatin is tagmented within cells using transposase (10X Genomics (10XG) kit). Polyadenylated mRNA is retained within the cells through Step1. Bottom panel: Step 2—cells are encapsulated and barcoded in gel bead-in-emulsions (GEMs) via 10XG Chromium instrument. The scHiC, scATAC, and scProtein modalities are barcoded using 10XG scATAC kit reagents, and the scRNA modality is barcoded using the 10XG Multiome kit reagents. Step 3-next generation sequencing (NGS) libraries are constructed. Depicted are NGS library molecules for each modality. For each library molecule type, the outer bars are sample-index containing NGS adapters, and the second bar from the left bar is a 10X barcode. For the accessible chromatin library molecule, the insert is an accessible chromatin fragment for scATAC. For the chromatin interaction library molecule, the chimeric insert is a chromatin interaction from an accessible region for scHiC. For the protein expression library molecule, the second bar from the right is an antibody derived tag (ADT) for scProtein. For the gene expression library molecule, the second bar from the right is cDNA, the third bar from the right is poly (dT), and the fourth bar from the right is a 10X unique molecular identifier (UMI) for scRNA. Step 4-paired-end NGS.

FIG. 9 shows images of multiplets (cell clumping) formed during one embodiment of a single-cell 3D gene regulation (sc3DGR) workflow.at varying concentrations of SDS. FIG. 9A is with no SDS used in the “Conditioning” step of the proximity ligation workflow. FIG. 9B is with 0.1% SDS at 62° C. for 5 min used in the “Conditioning” step of the proximity ligation workflow. FIG. 9C is with 0.25% SDS at 62° C. for 5 min used in the “Conditioning” step of the proximity ligation workflow. In FIGS. 9A-9C, the methods described below were modified as follows: no triton was used, cells were fixed, lysed then immediately to digestion master mix.

As shown in FIG. 9A, the use of 0% SDS in the “Conditioning” step of the proximity ligation workflow is compatible with proximity ligation, and also resulted in the best quality of nuclei in the PBMC experiments. The no SDS conditions has no clumping, which is important for single-cell analysis (to avoid dublets/multiplets when doing the droplet encapsulation step).

FIG. 10 shows Manhattan plots comparing various salt concentrations used in scATAC and scHiC modalities in one embodiment of the sc3DGR workflow using peripheral blood mononuclear cells (PBMCs). In Example 1, Applicants showed that increasing amounts of NaCl during the tagmentation reaction helped enable a highly specific chromatin accessibility signal in the Drop-C data, similar to that of scATAC-seq controls. These experiments were done using cultured cell lines. However, FIG. 10 shows results using primary human PBMCs and indicates that the prior NaCl optimization is not transferable to primary PBMCs. Even when using 100 mM NaCl, the specificity of the chromatin accessibility signal in the Drop-C data is poor and does not recapitulate scATAC-seq controls. In certain embodiments, NaCl concentration is increased during tagmentation. In other embodiments, the NaCl concentration is not increased during tagmentation.

The data in FIG. 10 reflects the method described below with the biotin pull-down. To make the plot, the ATAC data and the HiC data were combined. The units are normalized RPGC (per bin)=number of reads per bin/scaling factor for 1× average coverage.

FIG. 11 shows Manhattan plots comparing various length of library molecules used in one embodiment of the sc3DGR workflow using peripheral blood mononuclear cells (PBMCs). To help overcome the challenges with the poor specificity of the chromatin accessibility signal in the Drop-C data that was observed in the PBMC samples, it then was observed that the shortest library molecules are mostly derived from regions that are accessible (the true positive regions based on ATAC-seq data), whereas larger library molecules are more likely derived from regions that are not typically accessible (outside of the true positive regions, likely made accessible via the one or more proximity ligation steps that happen before tagmentation). Therefore, by bioinformatically analyzing only using the DNA sequence reads with insert sizes 0-147 bp, one can derive much more specific chromatin accessibility profiles in the Drop-C data. The data shown in FIG. 11 was generated using biotin pulldown protocol, no SDS and plotted data combines the ATAC and HiC data. The units are normalized RPGC (per bin)=number of reads per bin/scaling factor for 1× average coverage. FIG. 12 shows an analysis of sequence motifs enriched at the transposase insertion sites that are outside of the true positive accessible regions. To better understand the source of the non-specific tagmentation (and then to possibly correct/normalize against it), a tool called ChromBPNet (https://github.com/kundajelab/chrombpnet) was used to identify sequence motifs enriched at the transposase insertion sites that are outside of the true positive accessible regions. It was discovered that the restriction enzyme cut site motifs were strongly enriched at transposase insertion sites, suggesting that restriction digestion makes the DNA somewhat of “magnet” for transposition. Or in other words, it's possible that restriction digestion in regions not normally accessible to transposase, now become accessible to transposase. The mechanism is not clear. The columns in FIG. 12 from left to right are as follows: Pattern=top 1-10 most represented/enriched motif; NumSeqs=Number of observations; CWM_fwd/rev=cwm_fwd, cwm_rev are the forward and reverse complemented consolidated motifs from contribution scores in subset of random peaks.; Match0=Tn5_*=known Tn5 bias site; or known TF motifs; NaN=no known TF or TN5 site, which in certain embodiments is RE cut site; and Qvalue indicates basically how much an identified motif is similar to known Tn5 bias sequences and smaller values means that the sequences are closer to Tn5 bias sequences. The data shown in FIG. 12 was generated using biotin pulldown protocol, no SDS and plotted data uses only the ATAC library data.

FIG. 13 shows Manhattan plots of scATAC and scHiC modalities in one embodiment of the sc3DGR workflow where the scHiC is also subjected to filtering. To obtain the data in FIG. 13, the sequence of the non-peaks (Tn5 bias and RE cut site) were identified, then those features were regressed out. Apply to filter artificial peaks. The filtering happens at the Peak level (i.e. at the called peaks). MACS2 software was used for peak calling (default settings). The data shown in FIG. 13 were generated using biotin pulldown protocol, no SDS. The Drop C signal track and peak calling are from the ATAC library only.

FIG. 14 shows clustering of PBMCs using cell-surface protein expression profiles in one embodiment of the sc3DGR workflow. In FIG. 14, the top row data were generated using biotin pulldown protocol, no SDS and no salt. The middle row data were generated using biotin pulldown protocol, with 0.1% SDS and no salt. The bottom row data were generates using biotin pulldown protocol with 0.1% SDS and 100 mM NaCl. The clustering is based on ADT counts for 36 antibodies.

FIG. 15 is an enhanced view of the upper left image in FIG. 14B showing the cell type designation. FIG. 16 shows clustering of clustering of PBMCs using cell-surface protein expression profiles in one embodiment of the sc3DGR workflow. The data shown in FIG. 16 were generated using biotin pulldown protocol, no SDS. In FIG. 16 (left panel), accessibility clustering is shown using total peaks; all reads from ATAC library were used. In FIG. 17 (middle panel), peaks were filtered from chromBPnet; all reads from ATAC library were used. In FIG. 17 (right panel) is shown the clustering using the HiC library was from 50 KB size and entire genome and all pairwise interactions with >0 were used.

FIG. 17 shows HiC heat maps of chromatin interactions of the BCL11B encoding region in monocytes (FIG. 17A), T-cells (FIG. 17B), NK (FIG. 17C), and B-cells (FIG. 17D) in accordance with one embodiment of the sc3DGR workflow. The data shown in FIG. 17 were generated using biotin pulldown protocol, no SDS.

FIG. 18 shows HiC heat maps of chromatin interactions of the KLHL14 encoding region in monocytes (FIG. 18A), T-cells (FIG. 18B), NK (FIG. 18C), and B-cells (FIG. 18D) in accordance with one embodiment of the sc3DGR workflow. The data shown in FIG. 18 were generated using biotin pulldown protocol, no SDS.

FIG. 19 shows HiC heat maps of chromatin interactions of the SPI1 encoding region in monocytes (FIG. 19A), T-cells (FIG. 19B), NK (FIG. 19C), and B-cells (FIG. 19D) in accordance with one embodiment of the sc3DGR workflow. The data shown in FIG. 19 were generated using biotin pulldown protocol, no SDS.

FIG. 20 shows a TSS metaplot of the data shown in FIG. 11.

### Materials and Methods

The following materials and methods were used to perform certain assays described above. The following protocol is for a sc3DGR approach combining a Hi-C method with 10× Chromium SCATAC. The workflow described below includes biotinylation steps and targeted tagmentation. Other features may be included in other workflow configurations. The purpose of the sc3DGR workflow is to generate high quality chromatin accessibility profiles (comparable to scATAC-seq alone) combined with high quality chromatin 3D architecture information. Tables 13-28 provide materials used for certain aspects of the sc3DGR workflow.

TABLE 13
ADT/HTO staining buffer

| | Final | Stock | |
|---|---|---|---|
| Staining | concentration | Concentration | 20 ml |
| BSA | 2% | 10% | 4 ml |
| Tween | 0.01% | 10% | 20 μl |
| PBS | | | 16 ml |

TABLE 14
Wash buffer 10x stock

| | Working | Working | Working | Working |
|---|---|---|---|---|
| Wash Stock | Conc. | Conc. | Conc. | Conc. |
| Tris-HCl (pH 7.5) | 10 mM | 1M | 100 mM | 5 ml |
| NaCl | 10 mM | 5M | 100 mM | 1 ml |
| Tween | 0.1% | 10% | 1% | 5 ml |
| Water | | | | 39 ml |

TABLE 15
Lysis buffer 10x stock

| | Working | Reagent | 10x | 10 ml | |
|---|---|---|---|---|---|
| Lysis stock | conc. | stock conc. | stock conc. | 10x stock | |
| Tris-HCl (pH 7.5) | 10 mM | 1M | 100 mM | 1 | ml |
| NaCl | 10 mM | 5M | 100 mM | 200 | μl |
| MgCl₂ | 3 mM | 1M | 30 mM | 300 | μl |
| IGEPAL (NP40) | 0.1% | 10% | 1% | 1 | ml |
| Tween | 0.1% | 10% | 1% | 1 | ml |
| Water | | | | 6.5 | ml |

TABLE 16
Wash buffer I

| | reagent | |
|---|---|---|
| | stock conc. | 10 ml |
| wash buffer 10x stock | | 1 ml |
| bsa | 10% | 1 ml |
| water | | 8 ml |

TABLE 17
Lysis buffer

| OMNI lysis | Final Conc. | Stock Conc. | 2 ml | |
|---|---|---|---|---|
| Lysis Buffer 10x Stock | | 10x | 200 | μl |
| Digitonin | 0.01% | 10% | 4 | μl |
| BSA | 1% | 10% | 200 | μl |
| Water | | | 1.6 | ml |

TABLE 18
Wash Buffer 1

| Wash | Final Conc. | Stock Conc. | 20 ml | |
|---|---|---|---|---|
| Tris-HCl (pH 7.5) | 10 mM | 1M | 200 | μl |
| NaCl | 10 mM | 5M | 40 | μl |
| Tween | 0.1% | 10% | 200 | μl |
| BSA | 1% | 10% | 2 | ml |
| Water | | | 17.5 | ml |

TABLE 19
Lysis buffer

| OMNI lysis | Final Conc. | Stock Conc. | 2 ml | |
|---|---|---|---|---|
| Tris-HCl (pH 7.5) | 10 mM | 1M | 20 | μl |
| NaCl | 10 mM | 5M | 4 | μl |
| MgCl2 | 3 mM | 1M | 6 | μl |
| IGEPAL (NP40) | 0.1% | 10% | 20 | μl |
| Tween | 0.1% | 10% | 20 | ul |
| Digitonin | 0.01% | 5% | 4 | μl |
| BSA | 1% | 10% | 200 | μl |
| Water | | | 1.726 | ml |

TABLE 20
Antibodies used for cell surface protein expression

| TotalseqA | Catalog | Clone | rxn volume |
|---|---|---|---|
| CD10 | A0062 | Hl10a | 0.25 |
| CD19 | A0050 | H1B19 | 0.25 |
| CD20 | A0100 | 2H7 | 0.25 |
| CD33 | A0052 | | 0.25 |
| CD123 | A0064 | | 0.25 |
| CD11b | A0161 | ICRF44 | 0.25 |

The following protocols were used for certain aspects of the sc3DGR workflow.

### Cell Staining

1. Obtain all single cell suspensions (filter if needed) and measure viability and density (if viability is <90% it is encouraged to proceed with live cell enrichment).
2. Resuspend 1~2 million cells in 100 μl Staining buffer (2% BSA, 0.01% Tween in PBS).
3. Add 10 μl Fc Blocking reagent (FcX, BioLegend; mouse and human, respectively).
4. Incubate for 15 minutes on ice.
5. While cells are incubating in Fc Block, prepare antibody pool (panel or titrated amounts).
6. Aliquot the samples into 4 tubes (1 μl hashing antibody was added separately).
7. Incubate for 30 minutes on ice.
8. Wash cells three times with 1 ml staining buffer by centrifuging at 650 rcf for 5 min at 4° C.
9. Pool all samples together.
10. Add antibody-oligo pool to cells. (1.25 μg/ml per antibody).
11. Incubate for 30 minutes on ice.
12. Wash cells 3 times with 1 ml staining buffer by centrifuging at 650 g for 5 min at 4° C.
13. Resuspend cells in 1 ml PBS.

### Double Crosslinking

14. Add 8.5 ml PBS, 1 ml FBS, and 500 μl Formaldehyde.
15. Rotation for 10 min RT.
16. Add 500 μl 2.5 M Glycine to quench Formaldehyde crosslinking and rotate for 5 min RT (final concentration 125 mM).
17. Centrifuge at 650 rcf for 5 min.
18. Resuspend the cells in 1 ml PBS (1% BSA) and centrifuge at 650 rcf for 5 min.
19. Resuspend the cells in 900 μl PBS.
20. Add 100 μl 30 mM DSG for further crosslinking.
   Note that DSG should be diluted to 30 mM in DMSO (do not use 300 mM stock directly).
   300 mM DSG stock solution in DMSO (RT-50 mg, add 510.84 μl DMSO for 300 mM) (DSG: THERMO SCIENTIFIC™ (20593).
21. Incubate 40 min with rotation at RT.
22. Add 160 μl 2.5M Glycine and rotate for 5 min to quench the crosslinking (final concentration: 0.4 M glycine).
23. Resuspend the cells in 1 ml PBS (0.5% BSA) and centrifuge at 650 rcf for 5 min.
24. (Optional) Filter the cell using Flowmi Cell Strainer.

### Lysis

25. Resuspend in 100 μl (per million cells) chilled lysis buffer, pipette mix 10× and incubate for 10 min on ice (3 min for primary cells).
26. Add 1 ml Wash Buffer 1 (Table 18) to the lysed cells. Pipette mix 5x.
27. Centrifuge at 650 rcf for 5 min at 4° C.
28. Resuspend in 75 μl of 1× CutSmart buffer. For regular ATAC-seq, pellet cells and discard supernatant and resuspend in 100 μl of 10X GENOMICS™'s Nuclei buffer (1X) and store at 4° C. until proximity ligation is done.

### Conditioning

29. Add 25 μl of 1% SDS (final concentration: 0.25% SDS).
   Don't pipette mix or vortex-instead gently tap or invert the tube a few times to mix it.
   Some cells may be sensitive and damaged (ruptured) by 0.5% SDS; if that is the case, use 0.25% SDS but note that this could reduce digestion efficiency. In some embodiments, 0.1% SDS can be used.
30. Incubate tubes at 62° C. for 10 minutes in a PCR machine; 5 min for primary cells.
31. Add 10 μl of 10% Triton X-100 to SDS treated samples and incubate at 37° C. for 10 min.
   Don't pipette mix or vortex-instead gently tap or invert the tube a few times to mix it.
32. Add 200 μl Wash Buffer 1 (Table 18)+EDTA and incubate 1 min on ice.
   1 ml of Wash Buffer 1 (Table 18) and 20 μl of 0.5M EDTA (10 mM final concentration of EDTA)
33. Centrifuge at 350 rcf for 5 min at 4° C.
   In preferred embodiments, use 350 rcf for the first two centrifugations, but not over 600 rcf.
34. Remove supernatant and add 200 μl Wash Buffer I (Table 18).
35. Centrifuge at 350 rcf for 5 min at 4° C.
36. Remove supernatant and add 200 μl Wash Buffer | (Table 18).
37. Centrifuge at 650 rcf for 5 min at 4° C.
38. Carefully remove supernatant without disturbing the pellet.

### Digestion

39. Add 50 μl of a master mix containing the following reagents:

TABLE 21
Digestion (2 enzymes)

| | Volume per | 10% |
|---|---|---|
| Reagent | reaction | extra |
| Water | 24 | 15.4 |
| 10X CutSmart | 6 | 11 |
| Buffer (NEB) | | |
| Enzyme 1 (MBol) | 6 | 6.6 |
| Enzyme 2 (Dde1) | 6 | 6.6 |
| 7X Proteinase | 8 | 15.4 |
| inhibitor | | |
| cocktail | | |
| (EDTA free) | | |
| Total | 50 | |

40. Mix gently by pipetting, and incubate at 37° C. overnight (set the lid temperature to 42° C.). One may do interval shaking (900 rpm, 30 sec on, 4 min off) if Thermomixer is available (currentprotocals.onlinelibrary.wiley.com/doi/full/10.1002/cpz1.198), or mix by gentle pipetting 2 or 3 times, periodically.
41. Heat inactivation 10 min at 62° C.
42. Centrifuge at 650 rcf for 5 min at 4° C.
43. Carefully discard supernatant.
44. Resuspend sample in 200 μl of Wash Buffer 1 (Table 18).
45. Centrifuge at 650 rcf for 5 min at 4° C.
46. Carefully discard supernatant.
47. Gently add 200 μl of Wash Buffer I (Table 18).
48. Take 5-10% of samples to keep aside for Digestion QC (final volume 50 μl)
49. Centrifuge at 650 rcf for 5 min at 4° C.
50. Carefully remove supernatant without disturbing the pellet.

### Biotinylation

51. Resuspend the sample pellet in biotin fill-in mix (Table 21) and incubate the samples for 90 min at RT.

TABLE 21

| | Volume per | |
|---|---|---|
| Reagent | reaction | |
| 10X NEBuffer | 7 | ul |
| 10 mM dCTP | 1.5 | ul |
| 10 mM dGTP | 1.5 | ul |
| 10 mM dTTP | 1.5 | ul |
| 0.4 mM biotin | 37.5 | ul |
| 14-dATP | | |
| Protease | 10 | ul |
| Inhibitor | | |
| Klenow DNA | 10 | μl |
| polymerase | | |

52. Carefully discard supernatant.
53. Resuspend the sample in 200 μl of Wash Buffer 1 (Table 18)
54. Centrifuge at 650 rcf for 5 min at 4° C.
55. Carefully discard supernatant.
56. Resuspend the sample in 200 μl of Wash Buffer 1 (Table 18)
57. Pellet sample by centrifugation at 650×G at 4° C. for 5 min.
58. Carefully discard supernatant.

Ligation
66. Add 50 μl of a master mix containing the following reagents:

TABLE 22
Ligation

| | Volume per |
|---|---|
| Reagent | reaction |
| Water | 24 |
| T4 ligase buffer | 6 |
| T4 ligase (5 U/μl) | 12 |
| 7x Roche | 8 |
| complete, EDTA- | |
| free Protease | |
| inhibitor | |
| Total | 50 |

67. Mix gently by pipetting, and incubate at RT for 4 hours. Mix the sample solution by pipetting after 1 hour incubation, or incubate on the rotator.
68. Add 100 μl Wash Buffer 1 (Table 18).
69. Centrifuge at 650 rcf for 5 min and carefully remove supernatant.
70. Resuspend the sample in 200 μl Wash Buffer 1 (Table 18) and filter the cells using FLOWMI Cell Strainer.
71. Centrifuge at 650 rcf for 5 min and carefully remove supernatant.
72. Resuspend in 50 μl of 1X Nuclei Buffer (Table 23) and count the cells.

TABLE 23
1X Nuclei Buffer

| | Volume per |
|---|---|
| Reagent | reaction |
| Water | 890 ul |
| 20X Nuclei | 50 ul |
| Buffer (10X | |
| GENOMICS(TM)) | |
| 5M NaCl | 60 ul |

### Tagmentation

73. Prepare Transposition Mix (Table 24) on ice.

TABLE 24
Transposition Mix (10X GENOMICS(TM))

| | Volume per |
|---|---|
| Reagent | reaction |
| ATAC Buffer B | 7 ul |
| ATAC Enzyme | 3 ul |
| Cell Solution | ul |
| (300 mM NaCl) | |

74. Proceed according to 10X GENOMICS™ instructions (https://cdn. 10xgenomics.com/image/upload/v1666737555/support-documents/CG000209_Chromium_NextGEM_SingleCell_ATAC_ReagentKits_v1.1_UserG uide_RevG.pdf, last accessed Jun. 22, 2023) with following modifications:
   1. Make sure the tagmentation solution contains 100 mM NaCl.
   2. During the barcoding reaction (step 2.1) soike in 0.5 μl of 1 μM bridge oligo (there I no dead volume in the reaction, so final volume will be 65.5 μl for methods associated with Example 1 above and 60.5 μl for methods associated with Example
2).
   3. During GEM™ incubation (step 2.5), include a 5 min incubation at 40° C. at the beginning of the protocol, i.e.: 40° C. 5 min; 72° C. 5 min; 98° C. 30 sec; 98° C. 10 sec; 59° C. 30 sec, repeat 11× (total 12 cycles); 72° C. 1 min, 15° C. hold (this extra step is not essential when using TSA products, but increase efficiency in TSB and especially TSC tag capture).
   4. During silane bead elution (step 3.10_add 55.5 μl of Elution Solution 1 and subsequently recover ~55 μl. Keep 5 μl aside to use as input in the tag library PCR (2.5 μl for ADT and HTO) and with remaining 50 μl proceed to SPRI clean up as protocol.

### Tagmentation (Bulk Assay Only)

75. Resuspend 50,000 cells in 4 μl of nuclei buffer and mix with the tagmentation reagents as follows.
76. Prepare the tagmentation mix (Table 25) and incubate for 30 min.

TABLE 25
Tagmentation Mix

| | | |
|---|---|---|
| Reagent | 0 mM NaCl | 100 mM NaCl |

| | | | | |
|---|---|---|---|---|
| Sample | 10 | μl | 10 | μl |
| TD buffer | 15 | μl | 15 | μl |
| TDE | 3 | μl | 3 | μl |
| NaCl (5M) | 0 | μl | 0.6 | μl |
| Tween20 (10%) | 0.3 | ul | 0.3 | ul |
| Digitonin (1%) | 0.3 | ul | 0.3 | ul |
| Water | 1.4 | μl | 0.8 | μl |
| Total | 30 | ul | 30 | ul |

### Library Construction (Bulk Assay)

77. Follow the original ATAC protocol (currentprotocals.onlinelibrary.wiley.com/doi/10.1002/0471142727.mb2129s109).
78. Immediately following transposition, purify using a QIAGEN™ MINELUTE™ PCR Purification Kit.
   a. Add 280 μl of buffer ERC and spin down by centrifuge for 1 min at 13000g.
   b. Wash with 750 μl of Buffer PE and spin down.
   c. Dry with 1 min centrifuge.
   d. Add 10 μl elution buffer and incubate for 5 min.
79. To amplify transposed DNA fragments, combine the following in a PCR tube:
10 μl transposed DNA
   10 μl water
   2.5 μl of 25 UM barcoded PCR primer 1
   2.5 μl of 25 μM NEXTERA™ read2 primer (without barcode)
   25 μl NEB Next High-Fidelity 2X PCR Master Mix (NEW ENGLAND BIOLABS™ #M0541)
   In case of hot start NEB PCR master mix, incubate master mix at 98° C. for 1 min. After cooling down in ice, add to samples.
80. Terminal cycle as follows:
   1) 72° C., 5 min
   2) 98° C., 30 sec
   3) 98° C., 10 sec
   4) 59° C., 30 sec
   5) 72° C., 1 min
   6) Repeat steps 3-5, 4× (total 5 cycles)
   7) Hold at 4° C.

### Reverse Crosslinking

81. After step 3.1 in scATAC, elute DNA in 55 μl of elution solution 1 and put aside 5 μl of samples for ADT/HTO.
82. Proceed reverse crosslinking with 50 μl of samples. Can be done with QC samples in parallel.
83. Add 9 μl of a mastermix (Table 26) containing the following reagents to 50 μl of samples.

TABLE 26
Mastermix

| Reagent | |
|---|---|
| Buffer D (Arima | 2.5 |
| Genomics) | |
| Enzyme D | 6.5 |
| (Arima | |
| Genomics) | |

84. Add 5 μl of Buffer E (Arima Genomics) to the samples, mix gently by pipetting.
85. Incubate the samples in a PCR machine with the following condition:
   1) 55° C. 30 min
   2) 68° C., 90 min
   3) 25° C., 10 min
86. Add 100 μl of SPRI beads (2X) and incubate for 5 min.
87. Place on the magnet, high, until the solution clears.
88. Remove the supernatant and add 200 μl 80% ethanol to the pellet. Wait 30 sec.
89. Remove the ethanol.
90. Repeat the ethanol washing for a total of two washed.
91. Centrifuge briefly. Place on magnet, low.
92. Remove remaining ethanol.
93. Remove from the magnet. Immediately add 40.5 μl Buffer EB. Pipette mix.
94. Transfer 40 μl sample to a new tube strip.

### Biotin-Streptavidin Pulldown

95. If reverse crosslinking step is skipped, proceed with the following step just after step 3.1 of ScATAC v1.1 of 10X GENOMICS™.
96. Prepare for the biotin pull-down by washing 25 μl of streptavidin C-1 beads (per sample) in 1 ml of TWB buffer (Table 27).

TABLE 27
TWB

| Reagent | Final Conc. | Stock Conc. | 10 ml | |
|---|---|---|---|---|
| Tris-HCL, | 5 mM | 1M | 50 | ul |
| pH 7.5 | | | | |
| NaCl | 1M | 5M | 2 | ml |
| Tween | 0.05% | 10% | 50 | ul |
| EDTA | 0.5 mM | 0.5M | 10 | ul |
| Water | | | 7.89 | ul |

97. Mix by pipetting up and down 20 times.
98. Place tubes against a magnet and incubate 1 min or until solution is clear.
99. Discard supernatant and remove the tube from magnet.
100. Repeat washing steps two more times for a toral of three washes.
101. Resuspend beads in 40 μl of 2X binding buffer (Table 28).

TABLE 28
2X binding buffer

| Reagent | Total: 1 ml | |
|---|---|---|
| Tris-HCL, | 10 | ul |
| pH 7.5 | | |
| NaCl | 400 | ul |
| EDTA | 2 | ul |
| Water | 590 | ul |

102. Add to the samples and incubate at RT for 20 min with rotation.
103. Place sample against magnet and incubate until solution is clear.
104. Supernatant is ATAC library and should be collected. Transfer supernatant into a new tube and proceed to step 3.2 of 10 GENOMICS™ ATAC protocol with doubled volume of SPRI (for ASAP-Seq, save supernatant for ADT/HTO).
105. Wash beads by resuspending in 100 μl of TWB (Table 27) and incubate at 55° C. for 2 min. Set lid temperature to 85° C.
106. Place sample against magnet and incubate until solution is clear.
107. Discard supernatant and remove sample from magnet.
108. Wash beads by resuspending in 100 μl of TWB (Table 27) and incubate at 55° C. for 2 min. Set lid temperature to 85° C.
109 Place sample against magnet and incubate until solution is clear.
110 Discard supernatant and remove sample from magnet.
111. Wash beads by resuspending in 100 μl Elution Buffer.
112. Discard supernatant and remove sample from magnet.
113. Elute the beads in 20 μl of Elution Buffer.

### Library Construction (Bulk Assay Only)

114. For determining PCR cycles, do test PCR reaction as follows:
   5 μl 5 cycles PCR amplified DNA
   1.5 μl H₂O
   1.25 μl of 5 μM PCR primer 1 (⅕ diluted)
   1.25 μl of 5 μM PCR barcoded primer 2 (⅕ diluted)
   1 μl of 10X SYBR ( 1/10 diluted)
   5 μl of NEB PCR master mix
115. Thermal cycle as follows
   1) 98° C., 30 sec
   2) 98° C., 10 sec
   3) 63° C., 30 sec
   4) 72° C., 1 min
   5) repeat steps 2-4, 19× (total 20 cycles)
   6) Hold at 4° C.
116. Calculate the number of cycles that corresponds to ¼ of maximum fluorescent intensity.
117. Run the remaining 45 μl PCR reaction to the correct number of cycles.
   1) 98° C., 30 sec
   2) 98° C., 10 sec
   3) 63° C., 30 sec
   4) 72° C., 1 min
   5) repeat steps 2-4, x times
   6) Hold at 4° C.
118. Vortex to resuspend SPRISELECT reagent. Add 40 μl SPRISELECT reagent to each sample. Pipette mix.
119. Incubate 5 min at room temperature.
120. Place on the magnet⊕ High until the solution clears.
121 Transfer 130 μl supernatant to a new strip tube. DO NOT discard the supernatant.
122. Vortex to resuspend SPRISELECT reagent. Add 74 μl SPRISELECT reagent to each sample. Pipette mix.
123. Incubate 5 min at room temperature.
124. Place on the magnet⊕ High until the solution clears. Remove the supernatant. Add 200 μl 80% ethanol to the pellet. Wait 30 sec.
125. Remove the ethanol.
126 Repeat ethanol washing for a total of 2 washes.
127. Centrifuge briefly. Place on the magnet⊕ Low.
128. Remove remaining ethanol.
129 Remove from the magnet. Immediately add 20.5 μl Buffer EB. Pipette mix.
130 Incubate 2 min at room temperature.
131. Centrifuge briefly. Place on the magnet⊕ Low until the solution clears.
132. Transfer 20 μl sample to a new tube strip.
133. Store at 4° C. for up to 72 h or at −20° C. for long-term storage.

### Library Construction HTO/ADT

134. Transfer 20 μl sample to a new tube strip.
135. Store at 4° C. for up to 72 h or at −20° C. for long-term storage.
136. During SPRI cleanup (step 3.2d), save the supernatant. For the bead bound fraction proceed as per protocol. For the sup fraction, add 32 μl SPRI, let bind for 5 min. Collect beads on magnet, wash 2× with 80% EtOH, remove remaining ethanol and elute beads in 42 μl EB (or more if multiple indexing reactions need to take place, i.e., TSA and TSB products have been used). This can be combined with the 3 μl set aside after the silane purification, as input in the ADT/HTO indexing reaction:
PCR:
   50 μl 2× KAPA mix
   2.5 μl P5 10 μM
   2.5 μl RPxx (TSA family) or D7xx 10 μM
   (D7xx (Hashtags, TotalseqB), RPxx (TotalseqA))
   3-45 μl input fragments, water to 100 μl total
   (D701 was used for certain embodiments. For HTO, D7 primers can be used. For 3′ ADT panel, RP primers can be used.)

### Program:

95° C. 3 min
95° C. 20 sec
60° C. 30 sec, repeat ~13-15× (total 14-16 cycles)
72° C. 20 sec
72° C. 5 min
4° C. hold
14 cycles for this experiment.
64° C. for HTO and 60° C. for ADT (totalseq-A)
Expected product: 190 base pairs (bp)
137. Perform QC through gel running (2.5 μl), qubit and bioanalyzer.

### Library Construction Hi-C

138. PCR:
   a. 25 μl 2X KAPA mix
   b. 3.75 SI Primer B (10X GENOMICS™)
   c. 1.25 Single Index N Set A (10X GENOMICS™)
   d. 20 μl input fragments
   e. water to 100 μl total
139. Same PC condition with Sample Index PCR (Step 4.1 of scATAC). In certain embodiments, two more cycles than ATAC library are needed (e.g. if 5000 cells is targeted, 10 cycles ATAC library, 12 cycles for Hi-C library).
140. Follow the double size selection step (Step 4.2) of scATAC with modification of step e:
   a. Fill up to 100 μl with elution buffer
   b. Add 50 μl SPRIselect reagent in step e.

### Library Construction of ATAC

141. Follow the rest of the scATAC protocol of (10X GENOMICS™).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A method for preparing nucleic acid from single cells and/or single cell nuclei, comprising:
a) contacting a population of cells and/or cell nuclei with a first crosslinking agent and a second crosslinking agent, thereby generating a population of double-crosslinked cells and/or double-crosslinked cell nuclei;
b) contacting the population of cells and/or cell nuclei comprising double-crosslinked cells and/or double-crosslinked cell nuclei with one or more agents that preserve spatial-proximity relationships in the nucleic acid of the cells and/or cell nuclei; and
c) partitioning the population of cells and/or cell nuclei into partitions comprising partitioned single cells and/or partitioned single cell nuclei.

2. The method of claim 1, wherein (a) comprises:
(i) contacting the population of cells and/or cell nuclei with the first crosslinking agent, thereby generating a population of single-crosslinked cells and/or single-crosslinked cell nuclei;
(ii) contacting the population of single-crosslinked cells and/or single-crosslinked cell nuclei with the second crosslinking agent, thereby generating a population of double-crosslinked cells and/or double-crosslinked cell nuclei.

3. The method of claim 1, wherein the first crosslinking agent comprises formaldehyde or disuccinimidyl glutarate (DSG).

4. The method of claim 1, wherein the second crosslinking agent comprises formaldehyde or disuccinimidyl glutarate (DSG).

5. The method of claim 1, wherein the first crosslinking agent comprises formaldehyde and the second crosslinking agent comprises disuccinimidyl glutarate (DSG).

6. The method of claim 1, wherein (b) comprises contacting the population of cells and/or cell nuclei with one or more reagents that generate proximity ligated nucleic acid molecules.

7. The method of claim 1, wherein (b) comprises contacting the population of cells and/or cell nuclei with one or more restriction endonucleases.

8. The method of claim 7, wherein the one or more restriction endonucleases yield fragment sizes that average about 800 base pairs.

9. The method of claim 7, wherein (b) comprises contacting the population of cells and/or cell nuclei with one or more restriction endonucleases for more than 2 hours.

10. The method of claim 9, wherein (b) comprises contacting the population of cells and/or cell nuclei with one or more restriction endonucleases for more than 8 hours.

11. The method of claim 7, wherein (b) comprises contacting the population of cells and/or cell nuclei with two or more restriction endonucleases.

12. The method of claim 1, wherein (b) comprises contacting the population of cells and/or cell nuclei with a ligase.

13. The method of claim 1, wherein (b) comprises contacting the population of cells and/or cell nuclei with a junction marker.

14. The method of claim 1, further comprising after (b) and prior to (c), contacting the population of cells and/or cell nuclei with one or more agents that enrich for accessible chromatin.

15. The method of claim 14, wherein the one or more agents that enrich for accessible chromatin comprise a transposase.

16. The method of claim 15, wherein the transposase is a Tn5 transposase.

17. The method claim 14, wherein the one or more agents that enrich for accessible chromatin further comprise one or more oligonucleotides.

18. The method of claim 17, wherein the one or more oligonucleotides comprise sequencing primer sequences.

19. The method of claim 14, wherein the one or more agents that enrich for accessible chromatin are in a salt solution.

20. The method of claim 19, wherein the salt is present at a concentration of about 50 mM to about 200 mM.

21. The method of claim 19, wherein the salt is NaCl.

22. The method of claim 1, wherein the method further comprises prior to (a), contacting the population of cells and/or cell nuclei with a cell-surface protein binding agent.

23. The method of claim 22, wherein the binding agent comprises one or more antibodies.

24. The method claim 23, wherein one or more of the antibodies are conjugated to an oligonucleotide.

25. The method of claim 1, wherein the cells and/or cell nuclei are partitioned into droplets in (c).

26. The method of claim 25, wherein (c) comprises contacting the population of cells and/or cell nuclei with gel bead-in-emulsion (GEM) and one or more cell-specific and/or nucleus-specific barcode oligonucleotide species.

27. The method of claim 1, wherein the cells and/or cell nuclei are partitioned into wells in (c).

28. The method of claim 1, further comprising after (c), performing a heated crosslink reversal process.

29. The method of claim 28, further comprising after the heated crosslink reversal process, contacting cells and/or cell nuclei in the one or more partitions with an agent comprising a reverse transcriptase activity, thereby generating cDNA from mRNA present in the cells and/or cell nuclei in the one or more partitions.

30. The method of claim 1, further comprising after (c) generating one or more nucleic acid libraries from one or more of the cells and/or cell nuclei in the partitions.

31. The method of claim 30, wherein the one or more nucleic acid libraries comprise one or more nucleic acid library molecule species chosen from:
i) library molecules comprising accessible chromatin fragments;
ii) library molecules comprising accessible chromatin fragments and proximity ligated chromatin fragments;
iii) library molecules comprising oligonucleotides representing protein expression; and
iv) library molecules comprising cDNA fragments representing gene expression.

32. The method of claim 30, further comprising sequencing the one or more nucleic acid libraries, thereby generating sequence reads.

33. The method of claim 32, further comprising analyzing the sequence reads according to a sequence read analysis.

34. The method of claim 33, wherein the sequence read analysis comprises identifying spatial-proximity relationship information.

35. The method of claim 33, wherein the sequence read analysis comprises identifying accessible chromatin information.

36. The method of claim 33, wherein the sequence read analysis comprises identifying protein expression information.

37. The method of claim 33, wherein the sequence read analysis comprises identifying gene expression information.

38. The method of claim 1, wherein cells and/or cell nuclei in the partitions comprise proximally ligated chromatin contacts (PLCC).

39. The method of claim 38, further comprising determining an amount of proximally ligated chromatin contacts (PLCC) per cell and/or cell nucleus.

40. The method of claim 39, wherein the PLCC per cell and/or cell nucleus is greater than the PLCC per cell and/or cell nucleus obtained under conditions in which single-crosslinked cells and/or single-crosslinked cell nuclei are subjected to conditions that preserve spatial-proximity relationships.

41. The method of claim 39, wherein the PLCC per cell and/or cell nucleus is about 5,000 or greater.

42. The method of claim 39, further comprising determining a percentage of proximally ligated chromatin contacts (PLCC).

43. The method of claim 42, wherein the percentage of PLCC is greater than the percentage of PLCC obtained under conditions in which single-crosslinked cells and/or single-crosslinked cell nuclei are subjected to conditions that preserve spatial-proximity relationships.

44. The method of claim 1, wherein a subset of partitions comprise two or more cells and/or cell nuclei.

45. The method of claim 44, wherein:
the subset of partitions comprising two more cells and/or cell nuclei comprises partitions each associated with a single barcode; and
a multiplet rate is a fraction of barcodes associated with two or more cells and/or cell nuclei.

46. The method of claim 45, further comprising determining the multiplet rate.

47. The method of claim 46, wherein the multiplet rate is less than the multiplet rate obtained under conditions in which single-crosslinked cells and/or single-crosslinked cell nuclei are subjected to conditions that preserve spatial-proximity relationships.

48. The method of claim 47, wherein the multiplet rate is less than about 8%.

49. A kit comprising:
a) a first crosslinking agent;
b) a second crosslinking agent; and
c) one or more agents that preserve spatial-proximity relationships in nucleic acid of cells and/or cell nuclei.

50. The kit of claim 49, further comprising one or more agents for partitioning a population of cells and/or cell nuclei into partitions comprising partitioned single cells and/or partitioned single cell nuclei.

51. The kit of claim 49, wherein the first crosslinking agent comprises formaldehyde or disuccinimidyl glutarate (DSG).

52. The kit of claim 49, wherein the second crosslinking agent comprises formaldehyde or disuccinimidyl glutarate (DSG).

53. The kit of claim 49, wherein the first crosslinking agent comprises formaldehyde and the second crosslinking agent comprises disuccinimidyl glutarate (DSG).

54. The kit of claim 49, wherein the one or more agents that preserve spatial-proximity relationships in the nucleic acid of the cells and/or cell nuclei comprise one or more reagents that generate proximity ligated nucleic acid molecules.

55. The kit of claim 49, wherein the one or more agents in (c) comprise one or more restriction endonucleases.

56. The kit of claim 49, wherein the one or more agents in (c) comprise two or more restriction endonucleases.

57. The kit of claim 49, wherein the one or more agents in (c) comprise a ligase.

58. The kit of claim 49, wherein the one or more agents in (c) comprise a junction marker.

59. The kit of claim 49, further comprising one or more agents that enrich for accessible chromatin.

60. The kit of claim 59, wherein the one or more agents that enrich for accessible chromatin comprise a transposase.

61. The kit of claim 60, wherein the transposase is a Tn5 transposase.

62. The kit of claim 59, wherein the one or more agents that enrich for accessible chromatin further comprise one or more oligonucleotides.

63. The kit of claim 62, wherein the one or more oligonucleotides comprise sequencing primer sequences.

64. The kit of claim 59, wherein the one or more agents that enrich for accessible chromatin are in a salt solution.

65. The kit of claim 64, wherein the salt is present at a concentration of about 50 mM to about 200 mM.

66. The kit of claim 64, wherein the salt is NaCl.

67. The kit of claim 49, further comprising a cell-surface protein binding agent.

68. The kit of claim 67, wherein the binding agent comprises one or more antibodies.

69. The kit of claim 68, one or more of the antibodies are conjugated to an oligonucleotide.

70. The kit of claim 50, wherein the one or more agents for partitioning a population of cells and/or cell nuclei into partitions comprise gelbead-in-emulsion (GEM) and one or more cell-specific and/or nucleus-specific barcode oligonucleotide species.

71. The kit of claim 49, further comprising an agent comprising a reverse transcriptase activity.

72. The kit of claim 49, further comprising one or more agents for generating one or more nucleic acid libraries.

73. A method for preparing nucleic acid from single cells and/or single cell nuclei, comprising:
a) contacting a population of cells and/or cell nuclei with one or more antibodies conjugated to an oligonucleotide;
b) contacting the population of cells and/or cell nuclei with a first crosslinking agent and a second crosslinking agent, thereby generating a population of double-crosslinked cells and/or double-crosslinked cell nuclei, wherein the first crosslinking agent comprises formaldehyde and the second crosslinking agent comprises disuccinimidyl glutarate (DSG);
c) contacting the population of cells and/or cell nuclei comprising double-crosslinked cells and/or double-crosslinked cell nuclei with one or more agents that generate proximity ligated nucleic acid molecules in the nucleic acid of the cells and/or cell nuclei, wherein the one or more reagents comprise two or more restriction endonucleases and a ligase;
d) contacting the population of cells and/or cell nuclei with one or more agents that enrich for accessible chromatin, wherein the one or more agents comprise a Tn5 transposase and one or more oligonucleotides comprising sequencing primer sequences;
e) partitioning the population of cells and/or cell nuclei into droplet partitions comprising partitioned single cells and/or partitioned single cell nuclei, wherein the partitioning comprises contacting the population of cells and/or cell nuclei with gelbead-in-emulsion (GEM) and one or more cell-specific and/or nucleus-specific barcode oligonucleotide species;
f) performing a heated crosslink reversal process and contacting cells and/or cell nuclei in the one or more partitions with an agent comprising a reverse transcriptase activity, thereby generating cDNA from mRNA present in the cells and/or cell nuclei in the one or more partitions; and
g) generating one or more nucleic acid libraries from one or more of the cells and/or cell nuclei in the partitions, wherein the one or more nucleic acid libraries comprise:
i) library molecules comprising accessible chromatin fragments;
ii) library molecules comprising accessible chromatin fragments and proximity ligated chromatin fragments;
iii) library molecules comprising oligonucleotides representing protein expression; and
iv) library molecules comprising cDNA fragments representing gene expression.

74. The method of claim 73, wherein the one or more agents in (c) comprise a junction marker.
